(19) United States
(12) Patent Application Publication
(10) Pub. No.: US 2023/0372218 A1
(43) Pub. Date: Nov. 23, 2023

(54) TOPICAL FORMULATIONS USEFUL FOR IMPROVING HAIR HEALTH OR SCALP HEALTH OR TREATING OR PREVENTING HAIR LOSS

(71) Applicant: Nutraceutical Wellness Inc., New York, NY (US)

(72) Inventors: Giorgio DELL'ACQUA, Jersey City, NJ (US); Tess MARSHALL, Brooklyn, NY (US); Nicole TOWNSEND, New York, NY (US); Aleksander RICHARDS, New York, NY (US); Pamela SCOCA, Somers, NY (US)

(21) Appl. No.: 18/309,723

(22) Filed: Apr. 28, 2023

Related U.S. Application Data
(60) Provisional application No. 63/337,011, filed on Apr. 29, 2022.

(51) Int. Cl. A61K 8/44; A61K 8/9789; A61Q 5/00; A61Q 5/02; A61K 8/9728; A61K 8/99

(52) U.S. Cl. CPC A61K 8/44; A61K 8/9789; A61Q 5/002; A61Q 5/02; A61K 8/9728; A61K 8/99; A61K 2800/85; A61K 2800/592

(57) ABSTRACT

This disclosure relates to topical formulations (including scalp essences, scalp cleansers, and scalp masks) for balancing the scalp microbiome and uses thereof to improve hair health or scalp health or treat or prevent hair loss.

### CROSS-REFERENCE TO RELATED APPLICATIONS

The present application claims the benefit of U.S. Provisional Patent Application No. 63/337,011 filed Apr. 29, 2022, which is incorporated herein by reference in its entirety.

### FIELD

This disclosure relates to topical formulations (including scalp essences, scalp cleansers, and scalp masks) for balancing the scalp microbiome and uses thereof to treat hair conditions.

### BACKGROUND

The human body surface (skin and scalp) provides ecological niches for an abundance of diverse bacteria and fungi, composing a “microbiome.” Dysbiosis in the microbiome, in which the bacterial abundances are skewed, is linked to scalp and skin disorders, which highlights the role of the host-bacterial community interdependence. It is becoming increasingly apparent that the scalp creates a unique microenvironment for the colonization of bacteria, creating its own microbiome independent of the skin's microbiome.

There are limited examples of therapies and ingredients that can target the microbiome and attempt to rebalance the dysbiotic conditions, and in turn also positively impact the scalp/skin health. Due to the complex and dynamic nature of the microbiome, strategies for creating microbiome-targeted therapies are presently unknown. There is a current unmet need for topical products which promote a healthy, well-balanced, bacterial diversity on the scalp.

### SUMMARY

In one aspect, provided herein are compositions comprising about 0.1%-0.3% w/w sarcosine, about 0.01%-0.1% w/w of kunzea pomifera or an extract thereof, about 0.01%-0.1% w/w of syzygium luehmannii or an extract thereof, about 0.01%-0.1% w/w of tasmannia lanceolata or an extract thereof, about 0.01%-0.1% w/w of pisum sativum or an extract thereof, and about 0.01%-0.1% w/w of salvia hispanica or an extract thereof.

Also provided herein are methods for improving hair health or scalp health or treating or preventing hair loss in a subject in need of hair health or scalp health improvement or hair loss treatment or prevention, comprising applying to the scalp of the subject an effective amount of the composition.

In another aspect provided herein are compositions comprising about 0.01%-0.1% w/w alpha-glucan oligosaccharide, about 0.01%-0.1% w/w kunzea pomifera or an extract thereof, about 0.01%-0.1% w/w syzygium luehmannii or an extract thereof, and about 0.01%-0.1% w/w tasmannia lanceolata or an extract thereof.

Also provided herein are methods for improving hair health or scalp health or treating or preventing hair loss in a subject in need of hair health or scalp health improvement or hair loss treatment or prevention, comprising applying to the scalp of the subject an effective amount of the composition.

In another aspect, provided herein are compositions comprising about 1%-3% w/w alpha-glucan oligosaccharide, about 0.1%-0.3% w/w bacillus ferment filtrate or an extract thereof, about 0.1%-0.3% w/w opuntia ficus-indica or an extract thereof, and about 0.01%-0.1% w/w prunus persica gum.

Also provided herein are methods for improving hair health or scalp health or treating or preventing hair loss in a subject in need of hair health or scalp health improvement or hair loss treatment or prevention, comprising applying to the scalp of the subject an effective amount of the composition.

### BRIEF DESCRIPTION OF DRAWINGS

FIGS. 1A-1C show the effect of a scalp essence, scalp cleanser, and scalp exfoliating mask, respectively, on the growth (Cfu/mL as a % of control) of a co-culture of S. epidermidis (red) and S. aureus (blue), normalized to the control (100% growth).

FIGS. 2A-2C show the influence of a scalp essence, scalp cleanser, and scalp exfoliating mask, respectively, on the microbial diversity (CFU/mL as % of control) of key scalp microbes in a co-culture.

FIGS. 3A-3C show the growth behavior (as % of control) of individual microbes in the presence of a scalp essence, scalp cleanser, and scalp exfoliating mask, respectively.

FIG. 4 shows a clinical grading scale for pictures of the scalp that may be used to assess the efficacy of the methods described herein.

FIGS. 5A and 5B depict the sebum content measured on the subjects' scalps (N=37) over the course of a 14-day use period of sample Shampoo (washout), either for the whole cohort (FIG. 5A) or stratified by the dry group and the oily group (FIG. 5B)

FIGS. 6A and 6B display the subjects' (N=37) scalp pH measured over time with 14 days of use of sample Shampoo, either for the whole cohort (FIG. 6A) or stratified by the dry group and the oily group (FIG. 6B)

FIGS. 7A and 7B display the subjects' (N=37) scalp hydration, as measured by Corneometer, with 14 days of use of sample Shampoo, either for the whole cohort (FIG. 7A) or stratified by the dry group and the oily group (FIG. 7B).

FIGS. 8A and 8B depict the subjects' scalp flakiness, as measured by Expert Scoring via Dermatologist, with use of sample Shampoo over 14 days.

FIGS. 9A and 9B show the overall subjects' scalp sebum measurements changes from baseline after use of sample mask as determined by sebumeter (FIG. 9A) or expert grading (FIG. 9B).

FIG. 10 shows the scalp pH measured over time with 14 days of use of sample mask.

FIG. 11 shows scalp hydration as measured by corneometer with 14 days of use of the sample essence.

FIG. 12 displays the sebum content, as measured by Sebumeter, after 28 days of using sample essence.

FIG. 13 shows the scalp pH measured over 14 days of use of sample essence.

FIGS. 14A and 14B display shedding of hair, as measured by the hair count sum of 4 quadrants collected from a brush over 28 days of using the essence. FIG. 14A shows total hair counts, and FIG. 14B displays shedding of hair, as measured by the hair count with parameters: bulb, no bulb and total.

FIG. 15 shows the results of expert clinical grading, flakiness and redness after 28 days of using the essence.

FIG. 16 depicts the sebum content over time for the dry group and the oily group; dry and oily status determined by an Expert evaluator. Dry group sebum levels decreased 32% over the course of 14 days using sample Shampoo. Oily group (baseline sebum>50) decreased 37% over the course of 14 days using sample Shampoo.

FIG. 17 displays the change in pH over the course of 14 days of sample Shampoo use for the dry and oily groups; dry and oily group determined by Expert Evaluation. There was no change in scalp pH with use of the sample Shampoo (% change<2% overall).

FIG. 18 shows the scalp hydration, as measured by Corneometer, over time (14 days) with use of sample Shampoo for the dry and oily groups; dry and oily scalp groups determined by Expert Evaluator. The dry scalp group hydration improved 25% over the course of 14 days of sample Shampoo use. The oily scalp group improved 19% over the course of 14 days of sample Shampoo use.

FIG. 19 shows scalp flakiness as measured by an Expert Scorer (Dermatologist) over the course of 14 days using sample Shampoo for the dry and oily groups; dry and oily groups determined by Expert Evaluator. The dry group improved 74% and the oily group 83% in expert-graded scalp flakiness scoring after 14 days of using sample Shampoo.

### DETAILED DESCRIPTION

Without wishing to be bound by theory, it is believed that the compositions and methods herein encourage a diverse scalp microbiome, which can lead to a healthier scalp because of the mutualistic host-microbe relationship, and that therefore are useful for promoting a balanced, diverse microbial community on the scalp and improving hair health or scalp health or treating or preventing hair loss.

### Definitions

As used herein, the term “about” when immediately preceding a numerical value means ±up to 20% of the numerical value. For example, “about” a numerical value means ±up to 20% of the numerical value, in some embodiments, ±up to 19%, ±up to 18%, ±up to 17%, ±up to 16%, ±up to 15%, ±up to 14%, ±up to 13%, ±up to 12%, ±up to 11%, ±up to 10%, ±up to 9%, ±up to 8%, ±up to 7%, ±up to 6%, ±up to 5%, ±up to 4%, ±up to 3%, ±up to 2%, ±up to 1%, ±up to less than 1%, or any other value or range of values therein. In some embodiments, “about” a numerical value means ±up to 10% of the numerical value embodiments.

As used herein, a “subject” is a mammal, e.g., a dog, cat, gerbil, horse, sheep, goat or human.

### Compositions

Provided herein are compositions useful for improving hair health or scalp health or treating or preventing hair loss. Examples of improving hair health or scalp health include hydrating the hair or scalp, rebalancing the hair's or scalp's microenvironment, microbiome and/or pH, reducing sebum buildup on the hair or scalp, reducing scalp inflammation or irritation and reducing dandruff. In some embodiments, the compositions are formulated for use as a scalp mask. In some embodiments, the compositions are formulated for use as a scalp cleanser, e.g., shampoo. In some embodiments, the compositions are formulated for use as a scalp essence. Unless otherwise indicated, the amount of an ingredient present in a composition is described using weight for weight mass percentages (“% w/w”), which is the weight of the ingredient compared to weight of the total composition.

Provided herein are compositions that may be used to exfoliate and/or rebalance the scalp. In some embodiments, the compositions are useful as a scalp mask. An illustrative composition useful as a scalp mask is set forth in Table 1. Without wishing to be bound by theory, it is believed that the compositions help rebalance the scalp environment and thus contribute to healthier conditions for hair growth. Thus, in some embodiments, the compositions provided herein are able to reduce excess sebum and oil that builds up on the scalp. In some embodiments, the compositions hydrate the scalp skin. In some embodiments, a scalp mask leads to rebalancing of the scalp microenvironment and microbiome.

In some embodiments, the compositions provided herein comprise sarcosine. Without being bound by theory, sarcosine, an amino acid derivative, is believed to confer sebum-reducing and microbiome-modulation capabilities. By reducing the sebum content on an oily scalp, it is believed that sarcosine effectively reduces the nutrients available to lipophilic bacteria and yeasts, such as P. acnes and/or Malassezia species. Sarcosine can also increase the microbial diversity of scalp skin over time, which is believed to help build a healthier scalp environment for hair health, scalp health or treatment or prevention of hair loss. Sarcosine may be present in the compositions provided herein at a concentration of about 0.05%-0.5%, e.g., about 0.05%-0.1%, about 0.05%-0.2%, about 0.05%-0.3%, about 0.05%-0.4%, about 0.1%-0.2%, about 0.1%-0.3%, about 0.1%-0.4%, or about 0.1%-0.5%. In some embodiments, sarcosine is present in a scalp mask at a concentration of about 0.1%-0.3%.

In some embodiments the compositions provided herein comprise one or more plants; one or more plant parts, e.g., roots, leaves, stems, seeds, flowers or fruits, e.g., berries; or an extract thereof, e.g., a wild berry extract. The wild berries or extracts thereof (for example, a blend or mixture of kunzea pomifera or an extract thereof, syzygium luehmannii or an extract thereof and tasmannia lanceolata or an extract thereof) is believed to help increase hydration on the scalp and alleviate dry scalp conditions. The kunzea pomifera extract syzygium luehmannii extract, and/or tasmannia lanceolata extract may each be a fruit extract, a leaf extract, a root extract, a stem extract, a seed extract, a flower extract, or a combination thereof. In some embodiments, the kunzea pomifera extract syzygium luehmannii extract, and/or tasmannia lanceolata extract is a whole plant extract. The kunzea pomifera or an extract thereof, syzygium luehmannii or an extract thereof, and tasmannia lanceolata or an extract thereof may each be present in the compositions at a concentration of about 0.005%-0.5%, e.g., about 0.005%-0.01%, about 0.005%-0.02%, about 0.005%-0.03%, about 0.005%-0.04%, about 0.005%-0.05%, about 0.01%-0.02%, about 0.01%-0.03%, about 0.01%-0.04%, about 0.01%-0.05%, about 0.01%-0.1%, about 0.1%-0.2%, about 0.1%-0.3%, about 0.1%-0.4%, or about 0.1%-0.5%. In some embodiments, each of kunzea pomifera or an extract thereof, syzygium luehmannii or an extract thereof, and tasmannia lanceolata or an extract thereof is present in the compositions at a concentration of about 0.01%-0.1%. In some embodiments, kunzea pomifera or an extract thereof, syzygium luehmannii or an extract thereof, and tasmannia lanceolata or an extract thereof are present in approximately equal proportions.

In some embodiments, the compositions provided herein comprise a blend or mixture of pisum sativum (pea) or an extract thereof and salvia hispanica or an extract thereof. The blend or mixture of pisum sativum or an extract thereof and salvia hispanica or an extract thereof is believed to reduce the sebum content on scalp and trans-epidermal water loss, thus increasing scalp hydration. The pisum sativum extract and/or the salvia hispanica extract may each be a fruit extract, a leaf extract, a root extract, a stem extract, a seed extract, a flower extract, or a combination thereof. In some embodiments, the pisum sativum extract and/or the salvia hispanica extract is a whole plant extract. Pisum sativum or an extract thereof and salvia hispanica or an extract thereof may each be present in the composition at a concentration of about 0.005%-0.5%, e.g., about 0.005%-0.01%, about 0.005%-0.02%, about 0.005%-0.03%, about 0.005%-0.04%, about 0.005%-0.05%, about 0.01%-0.02%, about 0.01%-0.03%, about 0.01%-0.04%, about 0.01%-0.05%, about 0.01%-0.1%, about 0.1%-0.2%, about 0.1%-0.3%, about 0.1%-0.4%, or about 0.1%-0.5%. In some embodiments, each of pisum sativum or an extract thereof and salvia hispanica or an extract thereof is present in a composition at a concentration of about 0.01%-0.1%. In some embodiments, pisum sativum or an extract thereof and salvia hispanica or an extract thereof are present in approximately equal proportions.

TABLE 1
Illustrative Composition Useful As A Scalp Mask

| | % Content | Illustrative | Illustrative |
|---|---|---|---|
| Ingredient | (w/w) | CAS | EINECS |
| Water | >30%* | 7732-18-5 | 231-791-2 |
| Glycerin | 3%-10% | 56-81-5 | 200-289-5 |
| Cetearyl Alcohol | 1%-3% | 67762-27-0 | 267-008-6 |
| Brassicamidopropyl Dimethylamine | 1%-3% | N/A | N/A |
| Jojoba Esters, Simmondsia chinensis or an | 1%-3% | N/A | N/A |
| extract thereof | | | |
| Parfum (Fragrance) | 1%-3% | N/A | N/A |
| Polyquaternium-37 | 0.3%-0.99% | 26161-33-1 | N/A |
| Lactic Acid | 0.3%-0.99% | 50-21-5 | 200-018-0 |
| Sodium Benzoate | 0.3%-0.99% | 532-32-1 | 208-534-8 |
| Vaccinium Myrtillus Extract | 0.1%-0.3% | 84082-34-8 | 281-983-5 |
| Piroctone Olamine | 0.1%-0.3% | 68890-66-4 | 272-574-2 |
| Potassium Sorbate | 0.1%-0.3% | 24634-61-5 | 246-376-1 |
| Saccharum Officinarum (Sugarcane) Extract | 0.1%-0.3% | 91722-22-4 | 294-424-5 |
| Sarcosine | 0.1%-0.3% | 107-97-1 | 203-538-6 |
| Tetrasodium Glutamate Diacetate | 0.01%-0.1% | 51981-21-6 | 257-573-7 |
| Montmorillonite | 0.01%-0.1% | 1318-93-0 | 215-288-5 |
| Citrus Aurantium Dulcis (Orange) Extract | 0.01%-0.1% | 8028-48-6 | 232-433-8 |
| Citrus Limon (Lemon) Extract | 0.01%-0.1% | 92346-89-9 | 296-174-2 |
| CI 77289 (Chromium Hydroxide Green) | 0.01%-0.1% | 12001-99-9 | N/A |
| Acer Saccharum (Sugar Maple) Extract | 0.01%-0.1% | 91770-22-8 | 294-807-7 |
| Isopropyl Alcohol | 0.01%-0.1% | 67-63-0 | 200-661-7 |
| Kunzea Pomifera Extract | 0.01%-0.1% | 1174757-73-3 | N/A |
| Syzygium Luehmannii Extract | 0.01%-0.1% | 1542151-08-5 | N/A |
| Tasmannia Lanceolata Extract | 0.01%-0.1% | 183815-52-3 | 920-166-6 |
| Pisum Sativum (Pea) Extract | 0.01%-0.1% | 90082-41-0 | 290-130-6 |
| Salvia Hispanica Extract | 0.01%-0.1% | N/A | N/A |
| Sodium Hydroxide | 0.01%-0.1% | 1310-73-2 | 215-185-5 |
| Tetrasodium EDTA | 0.01%-0.1% | 64-02-8 | 200-573-9 |
| Aspergillus Ferment | 0.01%-0.1% | N/A | N/A |

*may represent the remainder of the composition.

In some embodiments, the compositions provided herein comprise about 0.1%-0.3% sarcosine, about 0.01%-0.1% of kunzea pomifera or an extract thereof, about 0.01%-0.1% of syzygium luehmannii or an extract thereof, about 0.01%-0.1% of tasmannia lanceolata or an extract thereof, about 0.01%-0.1% of pisum sativum or an extract thereof, and about 0.01%-0.1% of salvia hispanica or an extract thereof.

In some embodiments, the compositions provided herein consist essentially of about 0.1%-0.3% sarcosine, about 0.01%-0.1% of kunzea pomifera or an extract thereof, about 0.01%-0.1% of syzygium luehmannii or an extract thereof, about 0.01%-0.1% of tasmannia lanceolata or an extract thereof, about 0.01%-0.1% of pisum sativum or an extract thereof, and about 0.01%-0.1% of salvia hispanica or an extract thereof.

In some embodiments, the compositions further comprise about 0.1%-0.3% of vaccinium myrtillus or an extract thereof, about 0.1%-0.3% of saccharum officinarum (sugarcane) or an extract thereof, about 0.01%-0.1% of citrus aurantium dulcis (orange) or an extract thereof, about 0.01%-0.1% of citrus limon (lemon) or an extract thereof, about 0.01%-0.1% of acer saccharum (sugar maple) or an extract thereof and about 1%-3% of jojoba esters, Simmondsia chinensis or an extract thereof. The vaccinium myrtillus extract, saccharum officinarum (sugarcane) extract, citrus aurantium dulcis (orange) extract, citrus limon (lemon) extract, and/or acer saccharum (sugar maple) extract may each be a fruit extract, a leaf extract, a root extract, a stem extract, a seed extract, a flower extract, or a combination thereof. In some embodiments, the vaccinium myrtillus extract, saccharum officinarum (sugarcane) extract, citrus aurantium dulcis (orange) extract, citrus limon (lemon) extract, and/or acer saccharum (sugar maple) extract is a whole plant extract.

In some embodiments, the compositions provided herein consist essentially of about 0.1%-0.3% sarcosine, about 0.01%-0.1% of kunzea pomifera or an extract thereof, about 0.01%-0.1% of syzygium luehmannii or an extract thereof, about 0.01%-0.1% of tasmannia lanceolata or an extract thereof, about 0.01%-0.1% of pisum sativum or an extract thereof, about 0.01%-0.1% of salvia hispanica or an extract thereof, about 0.3% of vaccinium myrtillus or an extract thereof, about 0.1%-0.3% of saccharum officinarum (sugarcane) or an extract thereof, about 0.01%-0.1% of citrus aurantium dulcis (orange) or an extract thereof, about 0.01%-0.1% of citrus limon (lemon) or an extract thereof, about 0.01%-0.1% of acer saccharum (sugar maple) or an extract thereof and about 1%-3% of jojoba esters, Simmondsia chinensis or an extract thereof.

In some embodiments, the compositions provided herein further comprise glycerin, cetearyl alcohol, brassicamidopropyl dimethylamine, parfum (fragrance), polyquaternium-37, lactic acid, sodium benzoate, piroctone olamine, potassium sorbate, tetrasodium glutamate diacetate, montmorillonite, CI 77289 (chromium hydroxide green), isopropyl alcohol, sodium hydroxide, tetrasodium EDTA, and/or aspergillus ferment.

Glycerin may be present in the compositions at a concentration of about 1%-20%, in some embodiments about 3%-10%.

Cetearyl alcohol, brassicamidopropyl dimethylamine, jojoba esters, Simmondsia chinensis or an extract thereof, and/or parfum (fragrance) may each be present in the compositions at a concentration of about 0.5%-5%, in some embodiments about 1%-3%.

Polyquaternium-37, lactic acid, and/or sodium benzoate may each be present in the compositions at a concentration of about 0.1%-1%, in some embodiments about 0.3%-0.99%.

Vaccinium myrtillus or an extract thereof, piroctone olamine, potassium sorbate, and/or saccharum officinarum (sugarcane) or an extract thereof may each be present in the compositions at a concentration of about 0.05%-0.5%, in some embodiments about 0.1%-0.3%.

Tetrasodium glutamate diacetate, montmorillonite, citrus aurantium dulcis (orange) or an extract thereof, citrus limon (lemon) or an extract thereof, CI 77289 (chromium hydroxide green), acer saccharum (sugar maple) or an extract thereof, isopropyl alcohol, sodium hydroxide, tetrasodium EDTA, and/or aspergillus ferment may each be present in the compositions at a concentration of about 0.005%-0.5%, in some embodiments about 0.01%-0.1%.

In some embodiments, the compositions (e.g., useful as a scalp mask) further comprise water. Water may form the base of the compositions. In some embodiments, water is present in concentrations of at least about 30%, and in concentrations of up to about 90%, up to about 95%, up to about 96%, up to about 97%, up to about 98%, or up to about 99%.

Provided herein are compositions which can be used to clean the scalp without destroying commensal bacteria. In some embodiments, such compositions are formulated for use as a scalp cleanser. An illustrative composition that is useful as a scalp cleanser is provided in Table 2. Without wishing to be bound by theory, it is believed that the compositions can gently clean the scalp without stripping commensal bacteria, creating a blank slate to gently reset the scalp conditions. Thus, in some embodiments, the compositions provided herein reset the scalp microenvironment. In some embodiments, the compositions formulated for use as a scalp cleanser are useful as a shampoo.

In some embodiments, the compositions provided herein comprise alpha-glucan oligosaccharide, a microbiome modulator prebiotic. Without being bound by theory, alpha-glucan oligosaccharide is believed to suppress the growth of commensal bacteria without stripping or strong antibacterial effects, thus increasing the presence of beneficial bacteria like staphylococcus epidermidis, and also increasing the biodiversity of the microbes. Alpha-glucan oligosaccharide may be present in the compositions at a concentration of about 0.005%-0.5%, e.g., about 0.005%-0.01%, about 0.005%-0.02%, about 0.005%-0.03%, about 0.005%-0.04%, about 0.005%-0.05%, about 0.01%-0.02%, about 0.01%-0.03%, about 0.01%-0.04%, about 0.01%-0.05%, about 0.01%-0.1%, about 0.1%-0.2%, about 0.1%-0.3%, about 0.1%-0.4%, or about 0.1%-0.5%. In some embodiments, alpha-glucan oligosaccharide is present in the compositions at a concentration of about 0.01%-0.1%.

In some embodiments the compositions (e.g., useful as a scalp cleanser) provided herein comprise one or more plants; one or more plant parts, e.g., roots, leaves, stems, seeds, flowers or fruits, e.g., berries; or an extract thereof, e.g., a wild berry extract. The blend or mixture of wild berries or an extract thereof (in some embodiments a blend or mixture of kunzea pomifera or an extract thereof syzygium luehmannii or an extract thereof, and tasmannia lanceolata or an extract thereof) is believed to help increase hydration on the scalp and alleviate dry scalp conditions. The kunzea pomifera extract syzygium luehmannii extract, and/or tasmannia lanceolata extract may each be a fruit extract, a leaf extract, a root extract, a stem extract, a seed extract, a flower extract or a combination thereof. In some embodiments, the kunzea pomifera extract syzygium luehmannii extract, and/or tasmannia lanceolata extract is a whole plant extract.

The kunzea pomifera or an extract thereof syzygium luehmannii or an extract thereof, and tasmannia lanceolata or an extract thereof may each be present in the compositions at a concentration of about 0.005%-0.5%, e.g., about 0.005%-0.01%, about 0.005%-0.02%, about 0.005%-0.03%, about 0.005%-0.04%, about 0.005%-0.05%, about 0.01%-0.02%, about 0.01%-0.03%, about 0.01%-0.04%, about 0.01%-0.05%, about 0.01%-0.1%, about 0.1%-0.2%, about 0.1%-0.3%, about 0.1%-0.4%, or about 0.1%-0.5%. In some embodiments, each of kunzea pomifera or an extract thereof syzygium luehmannii or an extract thereof, and tasmannia lanceolata or an extract thereof is present in the composition at a concentration of about 0.01%-0.1% In some embodiments, kunzea pomifera or an extract thereof syzygium luehmannii or an extract thereof, and tasmannia lanceolata or an extract thereof are present in approximately equal proportions.

TABLE 2
Illustrative Composition Useful As A Scalp Cleanser

| | % Content | Illustrative | Illustrative |
|---|---|---|---|
| Ingredient | (w/w) | CAS | EINECS |
| Water | >30%* | 7732-18-5 | 231-791-2 |
| Sodium C14-16 Olefin Sulfonate | 3%-10% | 68439-57-6 | 270-407-8 |
| Cocamidopropyl Betaine | 3%-10% | 97862-59-4 | 263-058-8 |
| Sodium Lauroy1 Sarcosinate | 3%-10% | 137-16-6 | 205-281-5 |
| Propanediol | 1%-3% | 504-63-2 | 207-997-3 |
| Sodium Chloride | 1%-3% | 7647-14-5 | 231-598-3 |
| Parfum (Fragrance) | 0.3%-0.99% | N/A | N/A |
| Cocamide MIPA | 0.3%-0.99% | 1335203-30-9 | 931-596-9 |
| Glycol Distearate | 0.3%-0.99% | 627-83-8 | 211-014-3 |
| Citric Acid | 0.3%-0.99% | 5949-29-1 | 201-069-1 |
| Hydroxy propyl Methylcellulose | 0.3%-0.99% | 9004-65-3 | N/A |
| Guar Hydroxypropyltrimonium Chloride | 0.3%-0.99% | 65497-29-2 | N/A |
| Glycerin | 0.3%-0.99% | 56-81-5 | 200-289-5 |
| Sodium Benzoate | 0.3%-0.99% | 532-32-1 | 208-534-8 |
| Polyquaternium-73 | 0.3%-0.99% | N/A | N/A |
| Caprylyl/Capryl Glucoside | 0.1%-0.3% | 68515-73-1 | N/A |
| Potassium Sorbate | 0.1%-0.3% | 24634-61-5 | 246-376-1 |
| Glycolipids | 0.1%-0.3% | N/A | N/A |
| Tetrasodium Glutamate Diacetate | 0.01%-0.1% | 51981-21-6 | 257-573-7 |
| Hydrolyzed Quinoa | 0.01%-0.1% | N/A | N/A |
| Diisopropyl Adipate | 0.01%-0.1% | 6938-94-9 | 230-072-0 |
| Sodium Hydroxide | 0.01%-0.1% | 1310-73-2 | 215-185-5 |
| Alpha-Glucan Oligosaccharide | 0.01%-0.1% | N/A | N/A |
| Triethyl Citrate | 0.01%-0.1% | 77-93-0 | 201-070-7 |
| Caprylyl Glyceryl Ether | 0.01%-0.1% | 10438-94-5 | N/A |
| Benzyl Alcohol | 0.01%-0.1% | 100-51-6 | 202-859-9 |
| Kunzea Pomifera Extract | 0.01%-0.1% | 1174757-73-3 | N/A |
| Syzygium Luehmannii Extract | 0.01%-0.1% | 1542151-08-5 | N/A |
| Tasmannia Lanceolata Extract | 0.01%-0.1% | 183815-52-3 | 920-166-6 |
| Aspergillus Ferment | 0.01%-0.1% | N/A | N/A |

*may represent the remainder of the composition.

In some embodiments, the compositions provided herein comprise about 0.01%-0.1% alpha-glucan oligosaccharide, about 0.01%-0.1% kunzea pomifera or an extract thereof, about 0.01%-0.1% syzygium luehmannii or an extract thereof, and about 0.01%-0.1% tasmannia lanceolata or an extract thereof.

In some embodiments, the compositions provided herein consist essentially of about 0.01%-0.1% alpha-glucan oligosaccharide, about 0.01%-0.1% kunzea pomifera or an extract thereof, about 0.01%-0.1% syzygium luehmannii or an extract thereof, and about 0.01%-0.1% tasmannia lanceolata or an extract thereof.

In some embodiments, the compositions further comprise about 0.01%-0.1% of hydrolyzed quinoa and about 0.01%-0.1% of aspergillus ferment.

In some embodiments, the compositions provided herein consist essentially of about 0.01%-0.1% alpha-glucan oligosaccharide, about 0.01%-0.1% kunzea pomifera or an extract thereof, about 0.01%-0.1% syzygium luehmannii or an extract thereof, about 0.01%-0.1% tasmannia lanceolata or an extract thereof, about 0.01%-0.1% of hydrolyzed quinoa and about 0.01%-0.1% of aspergillus ferment.

In some embodiments, the compositions further comprise sodium C14-16 olefin sulfonate, cocamidopropyl betaine sodium lauroyl sarcosinate, propanediol, sodium chloride, parfum (fragrance), cocamide mipa, glycol distearate, citric acid, hydroxypropyl methylcellulose, guar hydroxypropyltrimonium chloride, glycerin, sodium benzoate, polyquaternium-73, caprylyl/capryl glucoside, potassium sorbate, glycolipids, tetrasodium glutamate diacetate, diisopropyl adipate, sodium hydroxide, triethyl citrate, caprylyl glyceryl ether, and/or benzyl alcohol.

Sodium C14-16 olefin sulfonate, cocamidopropyl betaine, and/or sodium lauroyl sarcosinate may each be present in the compositions at a concentration of about 1%-20%, in some embodiments about 3%-10%.

Propanediol and/or sodium chloride may each be present in the compositions at a concentration of about 0.5%-5%, in some embodiments about 1%-3%.

Parfum (fragrance), cocamide mipa, glycol distearate, citric acid, hydroxypropyl methylcellulose, guar hydroxypropyltrimonium chloride, glycerin, sodium benzoate, and/or polyquaternium-73 may each be present in the compositions at a concentration of about 0.1%-1%, in some embodiments 0.3%-0.99%.

Caprylyl/capryl glucoside, potassium sorbate and/or glycolipids may each be present in the compositions at a concentration of about 0.05%-0.5%, in some embodiments about 0.1%-0.3%.

Tetrasodium glutamate diacetate, hydrolyzed quinoa, diisopropyl adipate, sodium hydroxide, triethyl citrate, caprylyl glyceryl ether, benzyl alcohol and/or aspergillus ferment may be present in in the compositions at a concentration of up to about 0.5%, in some embodiments up to about 0.1%.

In some embodiments, the compositions further comprise water. Water may form the base of the compositions. In some embodiments, water is present in concentrations of at least about 30%, and in concentrations of up to about 90%, up to about 95%, up to about 96%, up to about 97%, up to about 98%, or up to about 99%.

Provided herein are compositions which can be used to rebalance the scalp microbiome. In some embodiments, the compositions are formulated for use as a scalp essence. An illustrative composition useful as a scalp essence is set forth in Table 3. Without wishing to be bound by theory, it is believed that the compositions confer the ability to restore scalp and hair health. In particular, soothing, sebum-reducing, hydrating and microbiome-modulating ingredients are believed to contribute to a healthy, hydrated, restored scalp which in turn is believed to promote hair health. In some embodiments, the compositions provided herein result in rebuilding and restoring the scalp microenvironment conditions by modulating microbes, increasing hydration, reducing oiliness, reducing inflammation and/or soothing irritation. In some embodiments, the compositions are water-based. In some embodiments, the term “essence” is substantially similar to a “serum” and in such embodiments the terms are used interchangeably herein.

In some embodiments, the compositions provided herein comprise alpha-glucan oligosaccharide, a microbiome modulator prebiotic. Without being bound by theory, alpha-glucan oligosaccharide is believed to suppress the growth of commensal bacteria without stripping or strong antibacterial effects, thus increasing the presence of beneficial bacteria like Staphylococcus epidermidis, and also increasing the biodiversity of the microbes. Alpha-glucan oligosaccharide may be present in the compositions at a concentration of about 0.5%-5%, e.g., about 0.5%-1%, about 0.5%-2%, about 0.5%-3%, about 0.5%-4%, about 1%-2%, about 1%-3%, about 1%-4%, or about 1%-5%. In some embodiments, Alpha-glucan oligosaccharide is present in the compositions at a concentration of about 1%-3%.

In some embodiments the compositions provided herein comprise one or more plants; one or more plant parts, e.g., roots, leaves, stems, seeds, flowers or fruits, e.g., berries; or an extract thereof, e.g., a wild berry extract.

In some embodiments, the compositions provided herein comprises peach gum polysaccharides. Peach gum polysaccharides are believed to suppress lipophilic microbes like P. Acnes and M. Furfur, thus supplementing the microbiome modulation. The peach gum polysaccharides are further believed to confer a secondary benefit of soothing the scalp by reducing redness and visible inflammation. Peach gum polysaccharides may be present in the compositions at a concentration of about 0.005%-0.5%, e.g., about 0.005%-0.01%, about 0.005%-0.02%, about 0.005%-0.03%, about 0.005%-0.04%, about 0.005%-0.05%, about 0.01%-0.02%, about 0.01%-0.03%, about 0.01%-0.04%, about 0.01%-0.05%, about 0.01%-0.1%, about 0.1%-0.2%, about 0.1%-0.3%, about 0.1%-0.4%, or about 0.1%-0.5%. In some embodiments, peach gum polysaccharides are present in the compositions at a concentration of about 0.01%-0.1%.

In some embodiments the compositions provided herein comprises a bacillus ferment probiotic. The bacillus ferment postbiotic is believed to suppress the growth of pathogenic microbes, while also reducing sebum content and inflammation. Bacillus ferment postbiotic may be present in the compositions at a concentration of about 0.05%-0.5%, e.g., about 0.05%-0.1%, about 0.05%-0.2%, about 0.05%-0.3%, about 0.05%-0.4%, about 0.1%-0.2%, about 0.1%-0.3%, about 0.1%-0.4%, or about 0.1%-0.5%. In some embodiments, bacillus ferment postbiotic is present in the compositions at a concentration of about 0.1%-0.3%.

In some embodiments, the compositions provided herein comprises opuntia ficus-indica (prickly pear) or an extract thereof. Opuntia ficus-indica or an extract thereof is believed to complement the microbiome modulators as an additional soothing and hydrating component. The opuntia ficus-indica extract may be a fruit extract, a leaf extract, a root extract, a stem extract, a seed extract, a flower extract or a combination thereof. In some embodiments, the opuntia ficus-indica extract is a whole plant extract.

Opuntia ficus-indica or an extract thereof may be present in the compositions at a concentration of about 0.005%-0.5%, e.g., about 0.005%-0.01%, about 0.005%-0.02%, about 0.005%-0.03%, about 0.005%-0.04%, about 0.005%-0.05%, about 0.01%-0.02%, about 0.01%-0.03%, about 0.01%-0.04%, about 0.01%-0.05%, about 0.01%-0.1%, about 0.1%-0.2%, about 0.1%-0.3%, about 0.1%-0.4%, or about 0.1%-0.5%. In some embodiments, opuntia ficus-indica or an extract thereof is present in the compositions at a concentration of about 0.01%-0.1%.

TABLE 3
Illustrative Composition Useful As A Scalp Essence

| | % Content | Illustrative | Illustrative |
|---|---|---|---|
| Ingredient | (w/w) | CAS | EINECS |
| Water | >30%* | 7732-18-5 | 231-791-2 |
| Glycerin | 1%-3% | 56-81-5 | 200-289-5 |
| Alpha-Glucan Oligosaccharide | 1%-3% | N/A | N/A |
| Propanediol | 1%-3% | 504-63-2 | 207-997-3 |
| Smithsonite Extract | 1%-3% | N/A | N/A |
| Caprylyl/Capryl Glucoside | 0.3%-0.99% | 68515-73-1 | N/A |
| Guar Hydroxy propyltrimonium Chloride | 0.3%-0.99% | 65497-29-2 | N/A |
| Sodium Benzoate | 0.3%-0.99% | 532-32-1 | 208-534-8 |
| Potassium Sorbate | 0.1%-0.3% | 24634-61-5 | 246-376-1 |
| Hydrolyzed Quinoa | 0.1%-0.3% | N/A | N/A |
| Parfum (Fragrance) | 0.1%-0.3% | N/A | N/A |
| Lactic Acid | 0.1%-0.3% | 50-21-5 | 200-018-0 |
| Bacillus Ferment Filtrate Extract | 0.1%-0.3% | N/A | N/A |
| Opuntia Ficus-Indica Extract | 0.1%-0.3% | 90082-21-6 | 290-109-1 |
| Zingiber Officinale (Ginger) Extract | 0.1%-0.3% | N/A | N/A |
| Tetrasodium Glutamate Diacetate | 0.01%-0.1% | 51981-21-6 | 257-573-7 |
| Diisopropyl Adipate | 0.01%-0.1% | 6938-94-9 | 230-072-0 |
| Prunus Persica (Peach) Gum | 0.01%-0.1% | 84012-34-0 | 281-678-7 |
| Triethyl Citrate | 0.01%-0.1% | 77-93-0 | 201-070-7 |
| Sodium Hydroxide | 0.01%-0.1% | 1310-73-2 | 215-185-5 |
| Benzyl Alcohol | 0.01%-0.1% | 100-51-6 | 202-859-9 |

*may represent the remainder of the composition.

In some embodiments, the compositions provided herein comprise about 1%-3% alpha-glucan oligosaccharide, about 0.1%-0.3% bacillus ferment filtrate or an extract thereof, about 0.1%-0.3% opuntia ficus-indica or an extract thereof, and about 0.01%- 0.1% prunus persica gum. In some embodiments, the compositions provided herein consist essentially of about 1%-3% alpha-glucan oligosaccharide, about 0.1%-0.3% bacillus ferment filtrate or an extract thereof, 0.1%-0.3% opuntia ficus-indica or an extract thereof, and about 0.01%-0.1% prunus persica gum.

In some embodiments, the compositions comprise about 0.1%-0.3% w/w sarcosine, about 0.01%-0.1% w/w of kunzea pomifera or an extract thereof, about 0.01%-0.1% w/w of syzygium luehmannii or an extract thereof, about 0.01%-0.1% w/w of tasmannia lanceolata or an extract thereof, about 0.01%-0.1% w/w of pisum sativum or an extract thereof, and about 0.01%-0.1% w/w of salvia hispanica or an extract thereof. In some embodiments, the compositions comprise about 0.01%-0.1% w/w alpha-glucan oligosaccharide, about 0.01%-0.1% w/w kunzea pomifera or an extract thereof, about 0.01%-0.1% w/w syzygium luehmannii or an extract thereof, and about 0.01%-0.1% w/w tasmannia lanceolata or an extract thereof. In some embodiments, the compositions comprise about 1%-3% w/w alpha-glucan oligosaccharide, about 0.1%-0.3% w/w bacillus ferment filtrate or an extract thereof, about 0.1%-0.3% w/w opuntia ficus-indica or an extract thereof, and about 0.01%-0.1% w/w prunus persica gum.

In some embodiments, the compositions further comprise about 1%-3% of smithsonite or an extract thereof. In some embodiments, the compositions provided herein consist essentially of about 1%-3% alpha-glucan oligosaccharide, about 0.1%-0.3% bacillus ferment filtrate or an extract thereof, 0.1%-0.3% opuntia ficus-indica or an extract thereof, about 0.01%-0.1% prunus persica gum about 1%-3% of smithsonite or an extract thereof.

In some embodiments, the compositions further comprise glycerin, propanediol, caprylyl/capryl glucoside, guar hydroxypropyltrimonium chloride, sodium benzoate, potassium sorbate, hydrolyzed quinoa, parfum (fragrance), lactic acid, zingiber officinale (ginger) or an extract thereof, tetrasodium glutamate diacetate, diisopropyl adipate, triethyl citrate, sodium hydroxide, and/or benzyl alcohol.

Glycerin, propanediol and/or smithsonite or an extract thereof may each be present in the compositions at a concentration of about 0.5%-5%, in some embodiments about 1%-3%.

Caprylyl/capryl glucoside, guar hydroxypropyltrimonium chloride, and/or sodium benzoate may each be preset in the compositions (e.g., useful as a scalp essence) at a concentration of about 0.1-1%, in some embodiments about 0.3%-0.99%.

Potassium sorbate, hydrolyzed quinoa, parfum (fragrance), lactic acid, and/or zingiber officinale or an extract thereof may each be present in the compositions (e.g., useful as a scalp essence) at a concentration of about 0.05%-0.5%, in some embodiments about 0.1%-0.3%. The zingiber officinale extract may be a fruit extract, a leaf extract, a root extract, or a combination thereof. In some embodiments, the zingiber officinale extract is a whole plant extract.

Tetrasodium Glutamate Diacetate, Diisopropyl Adipate, Triethyl Citrate, Sodium Hydroxide, and/or Benzyl Alcohol may each be present in the compositions at a concentration of about 0.05%-0.5%, in some embodiments about 0.01%-0.1%.

In some embodiments, the compositions further comprise water. In some embodiments, water is present in concentrations of at least about 30%, and in concentrations of up to about 90%, up to about 95%, up to about 96%, up to about 97%, up to about 98%, or up to about 99%.

Excipients and Carriers

In some embodiments, the compositions provided herein further comprise a cosmetically acceptable carrier or excipient. In some embodiments, the carrier is an alcohol-free carrier. In some embodiments, the carrier base fluids that may be used in the compositions include any carrier fluid or combination of excipients suitable for use in cosmetic and/or medicinal applications. For example, the compositions may comprise an aqueous carrier base fluid.

In some embodiments, the carrier base fluid may act as a solvent, carrier, diluent and/or dispersant for the constituents of the compositions, and may allow for the uniform application of the constituents to the surface of the skin at an appropriate dilution, e.g., topical application. The carrier base fluid may also facilitate penetration of the composition into the skin.

In some embodiments, the carrier base fluid comprises a lotion suitable for topical application. In such embodiment, the lotion may comprise carbomer, water, glycerin, isopropyl myristate, mineral oil, stearic acid, glycol stearate, cetyl alcohol, dimethicone, preservatives, triethanolamine, and the like, or combinations thereof.

In some embodiments, the carrier base fluid comprises a gel suitable for topical application. In such embodiment, the gel may comprise water, carbomer, glycerin, propylene glycol, preservatives, and the like, or combinations thereof.

Alternatively, the carrier base fluid may comprise the balance of the composition after considering the amount of the other components used.

In another embodiment, one or more of the ingredients of the compositions may be solubilized in a solubilizer prior to mixing in the carrier base fluid, such that these ingredients become soluble in the carrier base fluid. Nonlimiting examples of solubilizers suitable for use in the present disclosure include water, glycerin (e.g., vegetable glycerin), various esters, polyethylene glycol (PEG), derivatives thereof, or combinations thereof.

In some embodiments, the compositions may further comprise one or more inactive ingredients, such as one or more surfactants, co-solvents, and excipients or fillers (e.g., solid, semi-solid, liquid, etc.); emollients; delivery enhancers; circulation enhancers; antimicrobial agents; anti-inflammatory agents; foaming agents; carriers; diluents; binding agents (e.g., dextran); thickening agents; gelling agents; vitamins, retinoids, and retinols (e.g., vitamin B3, vitamin A, etc.); pigments; fragrances; sunscreens and sunblocks; anti-oxidants and radical scavengers (e.g., tocopheryl acetate or vitamin E acetate); organic hydroxy acids; exfoliants; skin conditioners (e.g., ethylhexylglycerin, hydrolyzed soy protein, glycol distearate, cyclopentasiloxane, quaternium-79 hydrolyzed keratin, propylene glycol, etc.); moisturizers; humectants (e.g., hydrolyzed soy protein, propylene glycol, etc.); ceramides, pseudoceramides; phospholipids, sphingolipids, cholesterol, glucosamine; pharmaceutically acceptable penetrating agents (e.g., n-decylmethyl sulfoxide, lecithin organogels, tyrosine, lysine, etc.); preservatives (e.g., phenoxyethanol, benzoic acid, dehydroacetic acid, polyaminopropyl biguanide, DMDM hydantoin or 1,3-bis(hydroxymethyl)-5,5-dimethylimidazolidine-2,4-dione, iodopropynyl butylcarbamate, iodopropynyl butylcarbonate, stearalkonium chloride, etc.); amino acids such as proline; pyrrolidone carboxylic acid, its derivatives and salts; saccharide isomerate; panthenol (i.e., provitamin of B5); buffers together with a base such as triethanolamine or sodium hydroxide; waxes, such as beeswax, ozokerite wax, paraffin wax; plant extracts, obtained from plants such as Aloe vera leaf, cornflower, witch hazel, elderflower, green tea (e.g., Camellia sinensis) leaf, grape (e.g., Vitis vinifera) seed, jojoba (e.g., Simmondsia chinensis) seed, tea tree (e.g., Malaleuca alternifolia) leaf, rosemary (e.g., Rosmarinus officinalis), henna, sunflower (e.g., Helianthus annuus) seed, wild soybean (e.g., Glycine soja), argan tree kernel or argan, cucumber, shiso, etc.; or combinations thereof. As will be appreciated by one of skill in the art viewing this disclosure, the selection and amount of optional ingredients may be varied.

In some embodiments, the compositions of the invention further comprise sodium C14-16 olefin sulfonate, cetearyl alcohol, cocamidopropyl betaine, brassicamidopropyl dimethylamine, propanediol, sodium lauroyl sarcosinate, parfum (fragrance), sodium chloride, lactic acid, piroctone olamine, caprylyl/capryl glucoside, guar hydroxypropyltrimonium chloride, diisopropyl adipate, tetrasodium glutamate diacetate, glycol distearate, triethyl citrate, benzyl alcohol, sodium benzoate, potassium sorbate, cocamide MIPA, sodium hydroxide, hydroxypropyl methylcellulose, polyquaternium-37, isopropyl alcohol, citral, geraniol, caprylyl glyceryl ether, limonene, tetrasodium EDTA, linalool, citric acid, or CI 77289 (chromium hydroxide green).

In some embodiments, the compositions provided herein comprise one or more of water, glycerin, camellia sinensis (green tea) leaf or an extract thereof, glycine, larix europaea wood or an extract thereof, sodium metabisulfite, zinc chloride, pisum sativum (pea) sprout or an extract thereof, alcohol, olea europaea (olive) leaf or an extract thereof, curcuma longa (turmeric) root or an extract thereof, equisetum arvense (horsetail) or an extract thereof, hippophae rhamnoides (sea buckthorn) fruit oil, laminaria saccharina (neptune kelp) or an extract thereof, lepidium meyenii (maca) root or an extract thereof, melaleuca alternifolia (tea tree) leaf oil, moringa oleifera (moringa) leaf or an extract thereof, panax ginseng (ginseng) root or an extract thereof, DL-panthenol, L-theanine, Melatonin, Niacinamide, sodium dehydroacetate, sodium hyaluronate, and phytic acid.

It will be apparent to a person of skill in the art that, in addition to the illustrative compositions described above, other compositions may be made or produced using the ingredients listed in Table 1, Table 2, and/or Table 3.

In some embodiments, the compositions described herein do not comprise a sulfate. In some embodiments, the compositions described herein do not comprise a paraben. In some embodiments, the compositions described herein do not comprise a silicone. In some embodiments, the compositions described herein do not comprise a phthalate. In some embodiments, the compositions described herein do not comprise a synthetic fragrance. In some embodiments, the compositions described herein do not comprise a synthetic hormone.

In some embodiments, the compositions described herein are essentially free of sulfates. In some embodiments, the compositions described herein are essentially free of parabens. In some embodiments, the compositions described herein are essentially free of silicones. In some embodiments, the compositions described herein are essentially free of phthalates. In some embodiments, the compositions described herein are essentially free of synthetic fragrances. In some embodiments, the compositions described herein are essentially free of synthetic hormones. “As used herein, “essentially free” of a particular ingredient means that the compositions comprise no more than about 1% w/w of the ingredient.

### Methods of Use

Also provided herein are methods of using the compositions described herein to improve hair health or scalp health or treat or prevent hair loss. An example of hair health is hair quality. Examples of treating hair loss include reducing or inhibiting hair loss. Accordingly, further provided are methods for improving hair health or scalp health or treating or preventing hair loss in a subject in need of hair health or scalp health improvement or hair loss treatment or prevention, comprising applying to the scalp of the subject an effective amount of a composition disclosed herein.

In one aspect, provided herein is a method of improving hair health in a subject in need of hair health improvement, comprising applying to the scalp of the subject an effective amount of a composition described herein.

Conditions

In addition to improving or hair health or scalp health or treating or preventing hair loss, the methods and compositions described here may also improve the appearance of hair of the scalp to which the composition is applied, e.g., by thickening the hair and improving the luster, condition and manageability of the hair.

Administration

The compositions described herein may be applied to a subject's hair or scalp using any suitable regime. The compositions described herein may be applied at least once a week, such as at least every two days, or at least once each day. For example, application may be twice per day. In general, application of the compositions described herein, may be continued indefinitely. Alternatively, the application may be repeated only for a limited period, e.g. several weeks or months. application may then be repeated for a similar period at a later date.

Most commonly, the area of the skin to which the compositions described herein is applied will be the scalp.

In some embodiments, the methods for treating or preventing hair loss comprises topical application of a composition described herein to the scalp or any other body area where treatment or prevention of hair loss is desirable. The compositions described herein may be useful in a wide variety of finished products, including pharmaceutical products and cosmetic products. The compositions described herein may be prepared, packaged, and labeled for treating or preventing hair loss or diminishing the hair loss process.

In some embodiments, the compositions described herein, are topically administered, e.g., applied to a subject's scalp, in the form of a solution, gel, lotion, cream, ointment, oil-in-water emulsion, water-in-oil emulsion, stick, spray, aerosol, paste, mousse, tonic, shampoo, liposome or other cosmetically and topically suitable form. In some embodiments, the compositions described herein are topically applied to an area to be treated, for example the scalp in humans, by spraying, dabbing, swabbing, rubbing, or combinations thereof.

In some embodiments, the compositions described herein are topically applied in the form of a scalp stimulator foam. In another embodiment, the compositions described herein are topically dispersed on the scalp in an aerosol form such as in a chlorofluorocarbon solvent, for delivery in spray form. The spray form may present some advantages including high loading, enhanced uptake, convenient application, and less matting the hair in the region of application. In such embodiments, the compositions described herein may remain on the scalp for a period of time of about 1 week, alternatively about 1 day, alternatively about 12 h, alternatively about 4 h, alternatively about 1 h, alternatively about 30 min, alternatively about 5 min, or alternatively about 1 min. The compositions described herein may be removed at any desired point in time by washing and/or rinsing the scalp. In some embodiments, the compositions are applied daily without rinsing after the application until the next regular hair wash. In some embodiments, the compositions are allowed to remain on the scalp for at least 6 hours, 12 hours, 18 hours, 24 hours, 36 hours, or 48 hours.

In some embodiments, the compositions, e.g., useful as a scalp cleanser, are topically applied to a subject's scalp and subsequently rinsed from the subject's scalp, e.g., using an aqueous rinsing agent such as water.

Accordingly, further provided herein are methods for improving hair health or scalp health or treating or preventing hair loss in a subject in need of hair health or scalp health improvement or hair loss treatment or prevention, comprising: applying to the scalp of the subject an effective amount of a composition disclosed herein and subsequently rinsing the composition from the subject's scalp.

In some embodiments, the compositions, e.g., useful as a scalp mask, are topically applied to a subject's scalp; allowed to remain on the scalp for at least about 5 to about 8 minutes, in some embodiments at least about 5 to about 10 minutes; and subsequently rinsed from the subject's scalp, e.g., using an aqueous rinsing agent such as water.

Accordingly, further provided herein are methods for improving hair health or scalp health or treating or preventing hair loss in a subject in need of hair health or scalp health improvement or hair loss treatment or prevention, comprising: applying to the scalp of the subject an effective amount of a composition disclosed herein; allowing the composition to remain on the subject's scalp for at least about 5 to about 10 minutes; and subsequently rinsing the composition from the subject's scalp.

In some embodiments, the compositions, e.g., useful as a scalp essence, are topically applied to a subject's scalp; allowed to remain on the scalp for at least about at least about 6 hours, in some embodiments at least about 12 hours, in some embodiments at least about 24 hours and in some embodiments at least about 48 hours; and subsequently rinsed from the subject's scalp, e.g., using an aqueous rinsing agent such as water.

Accordingly, further provided herein are methods for improving hair health or scalp health or treating or preventing hair loss in a subject in need of hair health or scalp health improvement or hair loss treatment or prevention, comprising: applying to the scalp of the subject an effective amount of a composition disclosed herein; allowing the composition to remain on the subject's scalp for at least about 6 to about 48 hours; and subsequently rinsing the composition from the subject's scalp.

In some embodiments, the compositions are topically administered at least on a daily, and in some embodiments a twice daily, basis for a period of time. For example, a user may topically administer the compositions directly to a balding area or other area where treatment or prevention of hair loss is desired by gently massaging the composition of the present disclosure into the desired area. This process may be repeated later the same day. In some embodiments, the compositions may be left on the scalp or other area where treatment or prevention of hair loss is desired between applications occurring on the same day or on different days. As will be appreciated by one skilled in the art with the help of this disclosure, the compositions may be topically applied/administered periodically on a routine basis. Generally, the compositions may be topically administered on a daily basis, although more frequent applications also may be used.

In some embodiments, the application of the compositions may continue for any suitable period of time. For example, within a few weeks to a few months of the initial application, a user may notice a reduction in hair loss. It should be appreciated that the frequency with which the composition should be applied can vary depending on the desired effect. In particular, the degree of cosmetic enhancement might vary directly with the total amount and the combination of ingredients of the compositions.

As will be appreciated by those of skill in the art with the help of this disclosure, other methods may be used to topically apply/administer the compositions described herein.

In some embodiments, the compositions described herein may be advantageously used to treat or prevent hair loss. For example, as disclosed herein, the compositions described herein may diminish and/or stop hair loss in a time period of from about 7 days to about 80 days, alternatively from about 10 days to about 28 days, or alternatively from about 14 days to about 21 days.

While not intending to be limited by theory, it is believed that the compositions described herein may advantageously treat or prevent hair loss in a time period of from about 4 weeks to about 20 weeks, alternatively from about 4 weeks to about 16 weeks, or alternatively from about 4 weeks to about 10 weeks.

Dosage of the compositions described herein can be dependent upon many factors including, but not limited to, the severity of the hair loss, the subject's age, general health and individual response to the composition. Accordingly, dosages of the compositions described herein can vary and be readily adjusted, depending on each subject's response.

The compositions described herein can be administered topically, transdermally or subcutaneously. One skilled in the art will recognize the advantages of certain routes of administration. In some embodiments the compositions are administered by subcutaneous injection.

Dosage forms for the topical or transdermal administration of the compositions described herein include powders, sprays, ointments, pastes, creams, lotions, gels, solutions, patches and inhalants.

The compositions described herein may be freely combined with each other. Thus, in some embodiments a composition useful as a scalp mask is administered in combination with a composition useful as a scalp cleanser. In some embodiments, a composition useful as a scalp mask is administered in combination with a composition useful as a scalp essence. In some embodiments, a composition useful as a scalp cleanser is administered in combination with a compositions useful as a scalp essence. In some embodiments, a composition useful as a scalp mask, a composition useful as a scalp cleanser, and a composition useful as a scalp essence are administered in combination. When used in combination, the compositions may be administered sequentially, concurrently, or simultaneously. When used sequentially, the compositions may be administered in any suitable order.

In some embodiments, the methods provided herein further comprise microneedling the subject's scalp. In some embodiments, the methods further comprise microneedling the subject's scalp prior to applying to the subject's scalp a composition described herein.

In some embodiments, the methods provided herein comprise applying to the scalp of a subject about 0.1%-0.3% w/w sarcosine, about 0.01%-0.1% w/w of kunzea pomifera or an extract thereof, about 0.01%-0.1% w/w of syzygium luehmannii or an extract thereof, about 0.01%-0.1% w/w of tasmannia lanceolata or an extract thereof, about 0.01%-0.1% w/w of pisum sativum or an extract thereof, about 0.01%-0.1% w/w of salvia hispanica or an extract thereof, about 0.1%-0.3% w/w of vaccinium myrtillus or an extract thereof, about 0.1%-0.3% w/w of saccharum officinarum (sugarcane) or an extract thereof, about 0.01%-0.1% w/w of citrus aurantium dulcis (orange) or an extract thereof, about 0.01%-0.1% w/w of citrus limon (lemon) or an extract thereof, about 0.01%-0.1% w/w of acer saccharum (sugar maple) or an extract thereof and about 1%-3% w/w of jojoba esters, Simmondsia chinensis or an extract thereof. In some embodiments, the methods further comprise applying to the scalp of the subject about 30% w/w water, about 3%-10% w/w glycerin, about 1%-3% w/w cetearyl alcohol, about 1%-3% w/w brassicamidopropyl dimethylamine, about 1%-3% w/w parfum (fragrance), about 0.3%-0.99% w/w polyquaternium-37, about 0.3%-0.99% w/w lactic acid, about 0.3%-0.99% w/w sodium benzoate, about 0.1%-0.3% w/w piroctone olamine, about 0.1%-0.3% w/w potassium sorbate, about 0.01%-0.1% w/w tetrasodium glutamate diacetate, about 0.01%-0.1% w/w montmorillonite, about 0.01%-0.1% w/w CI 77289 (chromium hydroxide green), about 0.01%-0.1%w/w isopropyl alcohol, about 0.01%-0.1% w/w sodium hydroxide, about 0.01%-0.1% w/w tetrasodium EDTA, and/or about 0.01%-0.1% w/w aspergillus ferment.

In some embodiments, the methods provided herein comprise applying to the scalp of a subject a kunzea pomifera extract, syzygium luehmannii extract, tasmannia lanceolata extract, pisum sativum extract, salvia hispanica extract, saccharum, officinarum extract, citrus aurantium dulcis extract, citrus limon extract, and/or the acer saccharum extract, wherein the kunzea pomifera extract, syzygium luehmannii extract, tasmannia lanceolata extract, pisum sativum extract, salvia hispanica extract, saccharum, officinarum extract, citrus aurantium dulcis extract, citrus limon extract, and acer saccharum extract is a fruit extract, a leaf extract, a root extract, or a combination thereof. In some embodiments, the methods comprise applying to the scalp of the subject a kunzea pomifera extract, syzygium luehmannii extract, tasmannia lanceolata extract, pisum sativum extract, salvia hispanica extract, saccharum, officinarum extract, citrus aurantium dulcis extract, citrus limon extract, or acer saccharum extract, wherein the kunzea pomifera extract, syzygium luehmannii extract, tasmannia lanceolata extract, pisum sativum extract, salvia hispanica extract, saccharum, officinarum extract, citrus aurantium dulcis extract, citrus limon extract, and acer saccharum extract is a whole plant extract.

In some embodiments, the methods provided herein further comprise rinsing the subject's scalp at least about 5 to about 10 minutes after the applying.

In some embodiments, (a) the applying is applying twice a week. In some embodiments, the applying is a first applying; (b) the method results in an improvement of hydration of the scalp of at least 10% within about 1 month, about 2 months, or about 3 months, after the first applying; and (c) the improvement is determined using a corneometer. In some embodiments, the applying is a first applying; (b) the method results in a reduction in sebum production on the scalp of at least 10% within about 1 month, about 2 months, or about 3 months after the first applying and (c) the reduction is determined using a sebumeter.

In some embodiments, the methods provided herein comprise applying to the scalp of a subject about 0.01%-0.1% w/w alpha-glucan oligosaccharide, about 0.01%-0.1% w/w kunzea pomifera or an extract thereof, about 0.01%-0.1% w/w syzygium luehmannii or an extract thereof, about 0.01%-0.1% w/w tasmannia lanceolata or an extract thereof, about 0.01%-0.1% w/w of hydrolyzed quinoa and about 0.01%-0.1% w/w of aspergillus ferment. In some embodiments, the methods further comprise applying to the scalp of the subject about 30% w/w water, about 3%-10% w/w sodium C14-16 olefin sulfonate, about 3%-10% w/w cocamidopropyl betaine, about 3%-10% w/w sodium lauroyl sarcosinate, about 1%-3% w/w propanediol, about 1%-3% w/w sodium chloride, about 0.3%-0.99% w/w parfum (fragrance), about 0.3%-0.99% w/w cocamide mipa, about 0.3%-0.99% w/w glycol distearate, about 0.3%-0.99% w/w citric acid, about 0.3%-0.99% w/w hydroxypropyl methylcellulose, about 0.3%-0.99% w/w guar hydroxypropyltrimonium chloride, about 0.3%-0.99% w/w glycerin, about 0.3%-0.99% w/w sodium benzoate, about 0.3%-0.99% w/w polyquaternium-73, about 0.1%-0.3% w/w caprylyl/capryl glucoside, about 0.1%-0.3% w/w potassium sorbate, about 0.1%-0.3% w/w glycolipids, about 0.01%-0.1% w/w tetrasodium glutamate diacetate, about 0.01%-0.1% w/w diisopropyl adipate, about 0.01%-0.1% w/w sodium hydroxide, about 0.01%-0.1% w/w triethyl citrate, about 0.01%-0.1% w/w caprylyl glyceryl ether, and/or about 0.01%-0.1% w/w benzyl alcohol.

In some embodiments, the methods provided herein comprise applying to the scalp of a subject a kunzea pomifera extract, syzygium luehmannii extract, and/or a tasmannia lanceolata extract, and wherein the kunzea pomifera extract, syzygium luehmannii extract, and/or the tasmannia lanceolata extract is a fruit extract, a leaf extract, a root extract, or a combination thereof. In some embodiments, the methods provided herein comprise applying to the scalp of the subject a kunzea pomifera extract, syzygium luehmannii extract, and/or a tasmannia lanceolata extract, and wherein the kunzea pomifera extract, syzygium luehmannii extract, and/or the tasmannia lanceolata extract is a whole plant extract.

In some embodiments, the methods further comprise rinsing the subject's scalp. In some embodiments, the applying is applying daily.

In some embodiments, (a) the applying is a first applying; (b) the methods result in an improvement of hydration of the scalp of at least 10% within about 1 month, about 2 months, or about 3 months, after the first applying; and (c) the improvement is determined using a corneometer. In some embodiments, (a) the applying is a first applying; (b) the methods result in a reduction in sebum production on the scalp of at least 10% within about 1 month, about 2 months, or about 3 months after the first applying; and (c) the reduction is determined using a sebumeter.

In some embodiments, the methods provided herein comprise applying to the scalp of a subject about 1%-3% w/w alpha-glucan oligosaccharide, about 0.1%-0.3% w/w bacillus ferment filtrate or an extract thereof, about 0.1%-0.3% w/w opuntia ficus-indica or an extract thereof, about 0.01%-0.1% w/w prunus persica gum, and about 1%-3% w/w smithsonite or an extract thereof. In some embodiments, the methods further comprise applying to the scalp of the subject about 30% w/w water, about 1%-3% w/w glycerin, about 1%-3% w/w propanediol, about 0.3%-0.99% w/w caprylyl/capryl glucoside, about 0.3%-0.99% w/w guar hydroxypropyltrimonium chloride, about 0.3%-0.99% w/w sodium benzoate, about 0.1%-0.3% w/w potassium sorbate, about 0.1%-0.3% w/w hydrolyzed quinoa, about 0.1%-0.3% w/w parfum (fragrance), about 0.1%-0.3% w/w lactic acid, about 0.1%-0.3% w/w zingiber officinale (ginger) or an extract thereof, about 0.01%-0.1% w/w tetrasodium glutamate diacetate, about 0.01%-0.1% w/w diisopropyl adipate, about 0.01%-0.1% w/w triethyl citrate, about 0.01%-0.1% w/w sodium hydroxide, or about 0.01%-0.1% w/w benzyl alcohol.

In some embodiments, the methods provided herein comprise applying to the scalp of a subject an opuntia ficus-indica extract and/or a zingiber officinale extract, and wherein the opuntia ficus-indica extract and/or the zingiber officinale extract is a fruit extract, a leaf extract, a root extract, or a combination thereof. In some embodiments, the methods provided herein comprise applying to the scalp of the subject a opuntia ficus-indica extract and/or a zingiber officinale extract, and wherein the opuntia ficus-indica extract and/or the zingiber officinale extract is a whole plant extract.

In some embodiments, the applying is applying daily. In some embodiments, the methods further comprise rinsing the subject's scalp at least 6 hours, 12 hours, 18 hours, 24 hours, 36 hours, or 48 hours after the applying.

In some embodiments, (a) the applying is a first applying; (b) the methods result in an improvement of hydration of the scalp of at least 10% within about 1 month, about 2 months, or about 3 months, after the first applying; and (c) the improvement is determined using a corneometer. In some embodiments, (a) the applying is a first applying; (b) the methods result in a reduction in sebum production on the scalp of at least 10% within about 1 month, about 2 months, or about 3 months after the first applying; and (c) the reduction is determined using a sebumeter.

In some embodiments, the methods provided herein comprise applying to the subject's scalp sodium C14-16 olefin sulfonate, cetearyl alcohol, cocamidopropyl betaine, brassicamidopropyl dimethylamine, propanediol, sodium lauroyl sarcosinate, parfum (fragrance), sodium chloride, lactic acid, piroctone olamine, caprylyl/capryl glucoside, guar hydroxypropyltrimonium chloride, diisopropyl adipate, tetrasodium glutamate diacetate, glycol distearate, triethyl citrate, benzyl alcohol, sodium benzoate, potassium sorbate, cocamide MIPA, sodium hydroxide, hydroxypropyl methylcellulose, polyquatemium-37, isopropyl alcohol, citral, geraniol, caprylyl glyceryl ether, limonene, tetrasodium EDTA, linalool, citric acid, or CI 77289 (chromium hydroxide green).

In some embodiments, the methods described herein do not comprise applying a sulfate to a subject's scalp. In some embodiments, the methods described herein do not comprise applying a paraben to a subject's scalp. In some embodiments, the methods described herein do not comprise applying a silicone to a subject's scalp. In some embodiments, the methods described herein do not comprise applying a phthalate to a subject's scalp. In some embodiments, the methods described herein do not comprise applying a synthetic fragrance to a subject's scalp. In some embodiments, the methods described herein do not comprise applying a synthetic hormone to a subject's scalp.

Treatment or Prevention of Hair Loss

The compositions described herein may be used to treat or prevent hair loss in any situation where treatment or prevention of hair loss is desired.

In some embodiments, a subject treated in accordance with the methods described herein has experienced hair loss associated with one or more conditions, including but not limited to: anagen effluvium, telogen effluvium, drug properties Alopecia, radiation therapy, poisoning, diffuse alopecia areata, alopecia areata, loose anagen syndrome, postoperative occipital alopecia, syphilis, traction alopecia (traction alopecia), early androgenetic alopecia, alopecia areata, tricholtillomania tinea capitis, resting hair loss, telogen gravidarum, chronic resting hair loss, early male onset alopecia, iron deficiency, malnutrition/dyspepsia, Hypothyroidism, hyperthyroidism, systemic lupus erythematosus, chronic renal failure, liver dysfunction, advanced malignancy, viral or bacterial infection, and male developmental alopecia.

In some embodiments, the hair loss treated or prevented by the methods described herein is associated with one or more conditions, including but not limited to: anagen effluvium, telogen effluvium, drug properties Alopecia, radiation therapy, poisoning, diffuse alopecia areata, alopecia areata, loose anagen syndrome, postoperative occipital alopecia, syphilis, traction alopecia (traction alopecia), early androgenetic alopecia, alopecia areata, tricholtillomania tinea capitis, resting hair loss, telogen gravidarum, chronic resting hair loss, early male onset alopecia, iron deficiency, malnutrition/dyspepsia, Hypothyroidism, hyperthyroidism, systemic lupus erythematosus, chronic renal failure, liver dysfunction, advanced malignancy, viral or bacterial infection, and male developmental alopecia.

In particular, the methods of the present disclosure are useful for treating or preventing male developmental alopecia, alopecia areata, alopecia in drug-induced alopecia (e.g. following cancer chemotherapy), and recovery of alopecia resulting from radiation therapy. In some embodiments, the condition is an ageing-related condition, e.g., ageing-related hair loss. In some embodiments, the condition (e.g., hair loss) is caused by a nutrient deficiency, a mineral deficiency, and/or a vitamin deficiency.

In some embodiments, the compositions described herein are useful to treat or prevent hair loss in humans. However, the compositions described herein are also useful to treat or prevent hair loss in non-human mammals. For example, the compositions described herein may be used with farm animals such as sheep, in which treatment or prevention of fur (hair) loss would exhibit an economic benefit. The compositions may also be used to treat or prevent hair loss in companion animals such as dogs, cats, gerbils, etc. The dosages useful to obtain this effect will fit within the guidelines described herein. Likewise, the compositions described herein may be administered using formulations typically used for veterinary applications, taking into account the type of animal being treated. Other applications of the compositions described herein to treat or prevent hair loss will become readily apparent to one skilled in the art based upon the disclosure of this application.

In an embodiment, the compositions described herein may advantageously treat or prevent hair loss from dormant and/or injured hair follicles, e.g., the compositions described herein may have a rejuvenating effect on the hair follicles. Methods of Assessing Efficacy

Methods of assessing the efficacy of the methods and compositions described herein for treating or preventing hair loss are known in the art and are described herein.

A straightforward method for assessing improvement of hair health or scalp health or treatment or prevention of hair loss is by taking a photograph of a test area of the skin before and after application a composition described herein. The skin, e.g., scalp, may optionally be shaved for this purpose. A photograph is taken. The composition described herein is then applied. A second photograph is then taken. The improvement of hair health or scalp health or treatment or prevention of hair loss may be quantified by counting any combination of: (a) number of hairs appearing; (b) length of hair appearing; (c) thickness of hair appearing; (d) straightness of hair appearing; (e) area of hair growth.

For example, improvement of hair health or scalp health or treatment or prevention of hair loss may be assessed in an individual. An individual to whom a composition described herein is administered may display diminished hair loss, as measured by any of the parameters described above, of at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 100% or more. This may be compared to hair loss in an individual to which the composition is not administered. The diminished hair loss may be assessed by the number or thickness of hair. Otherwise, it may be assessed by measuring the area of hair loss.

Clinical Scales

Any suitable clinical scale known in the art or described herein for measuring or assessing hair health, scalp health or hair loss may be used to measure the efficacy of the methods described herein. See Gupta and Mysorel, J Cutan Aesthet Surg. 2016 January-March; 9(1): 3-12, which is incorporated herein by reference in its entirety for examples of scales that may be used to assess the efficacy of the methods described herein.

For example, in some embodiments, the Hamilton-Norwood scale can be used to measure the efficacy of the methods described herein. The Hamilton-Norwood scale classifies the severity of male pattern baldness into 7 stages. It is widely used to assess baldness, although some issues with reproducibility have been reported (Guarrera et al., Int J Trichology. 2009 July-December; 1(2): 120-122). The mildest stage is stage 1, where there is no significant hair loss is or visibly receding. In stage 2, hair loss becomes visible around the temples, but is usually still not significant. Hair loss becomes significant in stage three, which is associated with recession of the hairline, which may appear like a V-shaped, U-shaped, or M-shaped pattern. Hair loss around the vertex of the head may also occur in stage 3, but becomes more apparent in stage 4. In stage five, the remaining hair is significantly thinner and recedes further, and in stage 6 the bald areas become connected. The most severe stage is stage 7, where only a band of usually fine or thing hair around the sides and back of the head remains.

In some embodiments, the methods described herein result in treatment or prevention of hair loss as measured on the Hamilton-Norwood scale. In some embodiments, the methods result in an improvement within about 1 week, about 2 weeks, about 3 weeks, about 4 weeks, about 5 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein, as measured by the Hamilton-Norwood scale.

In some embodiments, the methods of treatment described herein results in a slowing of hair loss as measured on the Hamilton-Norwood scale, e.g., a slowing of hair loss compared to the rate of hair loss prior to treatment. In some embodiments, a subject's score on the Hamilton-Norwood scale is maintained for at least about 3 months, at least about 4 months, at least about 5 months, at least about 6 months, at least about 7 months, at least about 8 months, at least about 9 month, at least about 10 months, at least about 11 months, at least about 12 months, at least about 15 months, at least about 18 months, at least about 2 years, at least about 3 years, at least about 4 years, or at least about 5 years after the first administration of a composition described herein. In some embodiments, the subject had previously experienced a decrease in score on the Hamilton-Norwood scale.

Alternatively or additionally, the Ludwig scale may be used to measure the efficacy of the methods described herein. The Ludwig scale is commonly used to classify the severity of female hair loss into three stages (see Ludwig, British Journal of Dermatology (1977) 97, 247). The mildest stage is stage 1, in which there may be thinning on the top of the head. The hair loss then proceeds through stage 2, where the scalp becomes visible, through stage 3, in which all hair at the crown may be lost.

In some embodiments, the methods described herein results in treatment or prevention of hair loss as measured on the Ludwig scale. In some embodiments, the methods result in an improvement within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 months, about 10 months, about 11 months, or about 12 months after the first administration of a composition described herein, as measured by the Ludwig scale.

In some embodiments, the methods of treatment described herein results in a slowing of hair loss as measured on the Ludwig scale. In some embodiments, a subject's score on the Ludwig scale is maintained for at least about 3 months, at least about 4 months, at least about 5 months, at least about 6 months, at least about 7 months, at least about 8 months, at least about 9 month, at least about 10 months, at least about 11 months, at least about 12 months, at least about 15 months, at least about 18 months, at least about 2 years, at least about 3 years, at least about 4 years, or at least about 5 years after the first administration of composition described herein.

Hair Pull Test

The hair pull test may be used to measure the efficacy of the methods described herein. To conduct the hair pull test, the assessor holds a bundle of about 50-60 hairs close to the scalp and then firmly pulls on the bundle using slow traction as the fingers slide down the hair shaft, avoiding a fast and forceful tug. The pulled hairs are then counted, and any broken hairs that were extracted are discarded. The test is generally performed at multiple areas of the scalp, e.g., at the vertex, the parietal areas, and/or the occipital areas. If more than 10% of the hairs in each bundle are removed from a scalp area, the hair pull test is considered positive, removal of less than 10% of hairs is generally considered normal shedding. See McDonald et al., 2017, J Am Acad. Dermatology; 76(3): 472-477. In some embodiments, the methods described herein results in treatment or prevention of hair loss as measured by the hair pull test. In some embodiments, the methods result in an improvement of at least 10% as determined by the hair pull test within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein. In some embodiments, the method results in an improvement of at least 25% as determined by the hair pull test within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein. In some embodiments, the method results in an improvement of at least 50% as determined by the hair pull test within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of composition described herein.

In some embodiments, the methods described herein result in a slowing of hair loss as measured by the hair pull test. In some embodiments, a subject's hair pull test score is maintained for at least about 3 months, at least about 4 months, at least about 5 months, at least about 6 months, at least about 7 months, at least about 8 months, at least about 9 month, at least about 10 months, at least about 11 months, at least about 12 months, at least about 15 months, at least about 18 months, at least about 2 years, at least about 3 years, at least about 4 years, or at least about 5 years after the first administration of a composition described herein.

Hair Tug Test

The hair tug test may be used to measure the efficacy of the methods described herein. The hair tug test is similar to the hair pull test, but also evaluates hair stability, as well as hair loss. The hair tug test involves grasping hair between the thumb and forefinger near its root and tugging with the other hand on the same strand at its distal part. The hair tug test is positive if the hair fractures, indicating hair shaft fragility (Xu et al. Frontiers in Medicine, 24 Jul. 2017, 4:112).

In some embodiments, the methods described herein result in treatment or prevention of hair loss or damage as measured by the hair tug test. In some embodiments, the method results in an improvement of at least 10% as determined by the hair tug test within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein. In some embodiments, the methods result in an improvement of at least 25% as determined by the hair tug test within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein. In some embodiments, the methods result in an improvement of at least 50% as determined by the hair tug test within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein.

In some embodiments, the methods of treatment described herein result in a slowing of hair loss or damage as measured by the hair tug test. In some embodiments, a subject's hair tug test score is maintained for at least about 3 months, at least about 4 months, at least about 5 months, at least about 6 months, at least about 7 months, at least about 8 months, at least about 9 month, at least about 10 months, at least about 11 months, at least about 12 months, at least about 15 months, at least about 18 months, at least about 2 years, at least about 3 years, at least about 4 years, or at least about 5 years after the first administration of a composition described herein.

Card Test

The Card Test may be used to measure the efficacy of the methods described herein. The card test is a simple way of counting hair strands by holding a card covered with felt of a color that contrasts with the hair of the subject against the scalp and counting the number of growing strands. It may also be used to determine thickness and health of hair.

In some embodiments, the methods described herein result in treatment or prevention of hair loss as measured by the card test. In some embodiments, the method results in an improvement of at least 10% as determined by the card test within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein. In some embodiments, the methods result in an improvement of at least 25% as determined by the card test within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein. In some embodiments, the methods result in an improvement of at least 50% as determined by the card test within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein.

In some embodiments, the methods described herein result in a slowing of hair loss as measured by the card test. In some embodiments, the number of new hair strands as measured by the card test is maintained for at least about 3 months, at least about 4 months, at least about 5 months, at least about 6 months, at least about 7 months, at least about 8 months, at least about 9 month, at least about 10 months, at least about 11 months, at least about 12 months, at least about 15 months, at least about 18 months, at least about 2 years, at least about 3 years, at least about 4 years, or at least about 5 years after the first administration of a composition described herein.

Trichometry

Trichometry may be used to measure the efficacy of the methods described herein. Versions of trichometry include cross sectional trichometry and the use of photos of the scalp and hair. The trichometric index is a measure of the growth and shedding of any hair that is greater than 30 μm in diameter. A bundle of hair from a 2×2 cm scalp area is placed in the cross-section trichometer, which measures the cross-sectional area of the hair bundle. It then displays the Trichometric Index—which equals to bundle cross-sectional area in mm² per cm² of scalp surface multiplied by 100. See Wikramanayake et al., Int J Trichology. 2012 October-December; 4(4): 259-264. The method may also be carried out digitally using a Folliscope® or a video dermatoscope which takes pictures of the scalp and hair and provides information about hair coverage, including the total number of hairs on the scalp and the diameter of each strand of hair. The resulting images may be graded according to, for example, the following scale:

| | | | | | | | | | |
|---|---|---|---|---|---|---|---|---|---|
| 0 | 1 | 2 | 3 | 4 | 5 | 6 | 7 | 8 | 9 |

Flakiness

| | | | |
|---|---|---|---|
| No flakiness | Mild flakiness | Moderate flakiness | Severe flakiness |

Redness

| | | | |
|---|---|---|---|
| No redness | Mild redness | Moderate redness | Severe redness |

In some embodiments, the methods described herein results in treatment or prevention of hair loss as measured by trichometric index as determined by cross-section trichometer, Folliscope®, or video dermatoscope. In some embodiments, the methods result in an improvement of the trichometric index of at least 10% as determined by cross-section trichometer, Folliscope®, or video dermatoscope within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein. In some embodiments, the methods result in an improvement of the trichometric index of at least 25% as determined by cross-section trichometer, Folliscope®, or video dermatoscope within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein. In some embodiments, the methods result in an improvement of the trichometric index of at least 50% as determined by cross-section trichometer, Folliscope®, or video dermatoscope within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein.

In some embodiments, the methods described herein result in a slowing of hair loss as measured by cross-section trichometer. In some embodiments, a subject's trichometric index as determined by cross-section trichometer, Folliscope®, or video dermatoscope is maintained for at least about 3 months, at least about 4 months, at least about 5 months, at least about 6 months, at least about 7 months, at least about 8 months, at least about 9 month, at least about 10 months, at least about 11 months, at least about 12 months, at least about 15 months, at least about 18 months, at least about 2 years, at least about 3 years, at least about 4 years, or at least about 5 years after the first administration of a composition described herein.

Shedding Score

The shedding score as determined by the shedding scale may be used to measure the efficacy of the methods described herein. The shedding scale aims to distinguish normal hair shedding from hair loss. The shedding scale is visual scale shows nine piles of hair of different lengths (shoulder, mid-back, and lower back), with each pile comprising increasing hair amounts (10, 50, 100, 200, 300, 400, 500, 600, and 700 hair strands). The subject is asked to estimate the amount of shedding after brushing. A score of 1-4 is considered normal shedding while a score of 5-9 is considered excessive shedding. See Martínez-Velasco et al., Dermatol Ther (Heidelb). 2017 March; 7(1): 155-165. Alternatively, a subject's hair may be combed onto a white paper, applying a predetermined number (e.g., 2, 3, or 4) strokes to a quadrant of hair. Hairs that fall onto the white surface may be counted and the counts compared over time.

In some embodiments, the methods described herein result in treatment or prevention of hair loss as measured by shedding score determined on the shedding scale. In some embodiments, the methods result in an improvement of the shedding score of at least 10% as determined on the shedding scale within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein. In some embodiments, the methods result in an improvement of the shedding score of at least 25% as determined on the shedding scale within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein. In some embodiments, the methods result in an improvement of the shedding score of at least 50% as determined on the shedding scale within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein.

In some embodiments, the methods described herein result in a slowing of hair loss as measured by shedding score determined on the shedding scale. In some embodiments, a subject's shedding score as determined on the shedding scale is maintained for at least about 3 months, at least about 4 months, at least about 5 months, at least about 6 months, at least about 7 months, at least about 8 months, at least about 9 month, at least about 10 months, at least about 11 months, at least about 12 months, at least about 15 months, at least about 18 months, at least about 2 years, at least about 3 years, at least about 4 years, or at least about 5 years after the first administration of a composition described herein.

Corneometer

The compositions described herein are believed to contribute to the hydration of the scalp. It is believed that hydration of the scalp improves hair health and scalp health. Hydration may be evaluated, for example, using a corneometer. The electrical properties of the skin and its capacitance are largely determined by the water content of the horny layer. The Corneometer is an instrument designed to measure changes in the capacitance of the skin resulting from changes in the degree of hydration. It is particularly sensitive to low hydration levels. The Corneometer expresses the capacitance of the skin surface in arbitrary units of skin hydration.

In some embodiments, the methods described herein results in an improvement of scalp hydration as measured by corneometer. In some embodiments, the methods result in an improvement of hydration of the scalp of at least 10% within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein, as determined by corneometer. In some embodiments, the method results in an improvement of hydration of the scalp of at least 25% within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein as determined by corneometer. In some embodiments, the method results in an improvement of hydration of the scalp of at least 50% within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein, as determined by corneometer.

Sebumeter

In some aspects, the compositions described herein result in a decrease in sebum production on the scalp. It is believed that a decrease in sebum production on the scalp improves hair health or scalp health or treats or prevents hair loss. Such a reduction in sebum may be measured using, for example, a sebumeter. The Sebumeter is a commercially available instrument designed to evaluate the sebum content of the skin in μg/cm². It photometrically measures the increase in the transparency of a special translucent plastic strip when it becomes coated with sebum. The plastic strip is approximately 0.1 mm thick and 64 mm² in area. It is housed in the portable head of the instrument. The strip is backed by a mirror, which presses it against the relevant skin with a fixed pressure of 10N by means of a spring. The instrument also contains a timing device that limits the time of contact that the strip with the skin to thirty (30) seconds. The transparency of the plastic strip is evaluated by means of a microprocessor and is read off a digital instrument directly as μg of sebum per square centimeter.

In some embodiments, the methods result in a reduction in sebum production on the scalp of at least 10% within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein, as determined by sebumeter. In some embodiments, the methods result in a reduction in sebum production on the scalp of at least 25% within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein, as determined by sebumeter. In some embodiments, the methods result in a reduction in sebum production on the scalp of at least 50% within about 1 week, about 2 weeks, about 3 weeks, about 4 weeks, about 6 weeks, about 1 month, about 2 months, about 3 months, about 4 months, about 5 months, about 6 months, about 7 months, about 8 months, about 9 month, about 10 months, about 11 months, or about 12 months after the first administration of a composition described herein, as determined by sebumeter.

pH Measurements

The pH of the scalp influence the type and number of bacteria that grow in the scalp microbiome. The skin or scalp pH is easily measurable using, for example, a skin pH meter. The Skin-pH-Meter PH 905 is a quick, easy, and economical tool to specifically measure the pH on the skin surface or the scalp. The measurement of the pH-value covers an important characteristic of an aqueous solution: its acidity or alkalinity. Surface hydration and sebum together form a hydrolipidic film on the skin, also called acid mantle as its pH values is in the slightly acid range, between approx. pH 4.0-5.5. In some embodiments, the methods and compositions described herein do not significantly alter the pH of the skin. In some embodiments, the methods and compositions described herein maintain the pH of the skin. As the hydrolipidic film is almost an aqueous solution, with the Skin-pH-Meter PH 905 highly precise, easy, and quick pH-measurements can be performed directly on the skin surface. The pH value varies depending on the area of skin. The measurement of the pH- value of the skin is done with a glass electrode. The probe consists of a reference electrode (outer glass shaft of the probe filled with electrolyte (KCl+AgCl). Inside, there is a second chamber with the measurement electrode in an internal buffer. The measurement electrode is in direct contact with the skin via the special flat glass membrane on the tip of the probe which can perfectly sit in the skin surface. The reference electrode is only in electrical contact with the skin over the diaphragm to equalize the potential. No substance interchange takes place here. The modern, high-quality electronics of the probe allow a very quick (1 s) and reliable measurement avoiding occlusion effects. The measurement is based on a high-quality combined electrode, where both H+ ion sensitive electrode and additional reference electrode are placed in one glass housing. It is connected to a probe handle containing the measurement electronics. The probe head is planar for measuring optimally on the skin surface at all sites. A single and continuous measurement possible.

Analysis of the Scalp Microbiome

The composition of the scalp microbiome may be determined by genetic sequencing. To this end, a swab sample may be taken from the center of the scalp (when parted down the middle) and processed for sequencing analysis. The analysis may sequence the microbiome composition on the scalp to examine the effect of the testing product on viability and colonization.

In some embodiments, the methods described herein increase the population or proportion of beneficial commensal bacteria such as Staphylococcus epidermidis on the scalp. In some embodiments, the methods described herein decrease the population or proportion of harmful bacteria, such as lipophilic bacteria and yeasts, such as P. acnes and Malassezia species. like P. acnes and M. furfur on the scalp. In some embodiments, the compositions and methods described herein prevent an overgrowth of harmful bacteria on the scalp.

Consumer Perception Questionnaire

The effects the compositions and methods described herein can also be determined by questioning the subject with regard to consumer perception and the efficacy of the test material following use. Illustrative consumer perception questionnaires are set forth in Example 2.

Articles of Manufacture

Further provided herein is an article of manufacture, e.g., a container, containing a composition described herein. In some embodiments, the container is a closed container. In some embodiments, the closed container is sealed. In some embodiments, the container is plastic. In some embodiments, the plastic is recycled plastic. The articles of manufacture can be packaged for retail distribution, in association with instructions advising the consumer how to use the product to treat or prevent hair loss.

Kits

In some embodiments, a kit may contain any combination of the compositions described herein. For example, in some embodiments a kit comprises composition useful as a scalp mask and a composition useful as a scalp cleanser. In some embodiments, a kit comprises a composition useful as a scalp mask and a composition useful as a scalp essence. In some embodiments, a kit comprises a composition useful as a scalp cleanser and a composition useful as a scalp essence. In some embodiments, a kit comprises a composition useful as a scalp mask, a composition useful as a scalp cleanser, or a composition useful as a scalp essence. In some embodiments, a kit comprises a composition useful as a scalp mask, a composition useful as a scalp cleanser, and a composition useful as a scalp essence. An example of a scalp cleanser is a shampoo.

### EXAMPLES

The examples described in this section are provided for illustration only, and are not intended to limit the claimed invention.

### Example 1: Effects of a Composition of Table 3, Composition of Table 2, and Composition of Table 1 on Microbial Growth and Diversity

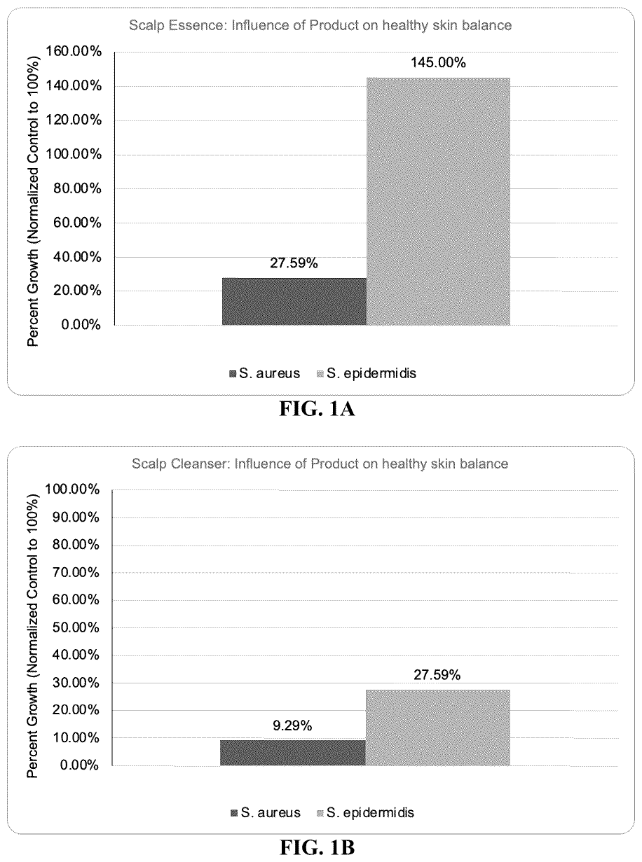

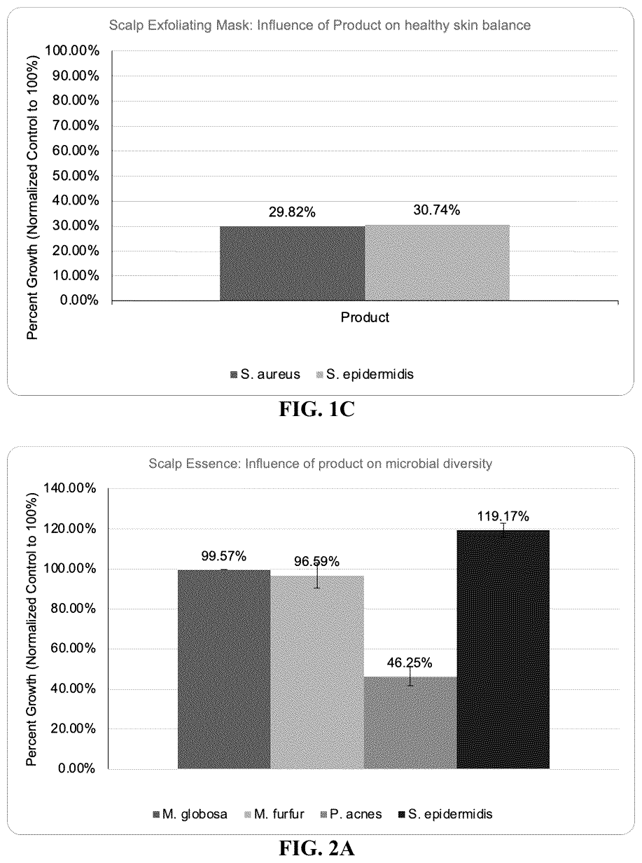

A co-culture of Staphylococcus epidermidis and Staphylococcus aureus was incubated for 15 minutes with: a composition of Table 3, comprising about 1%-3% alpha-glucan oligosaccharide, about 0.1%-0.3% bacillus ferment filtrate or an extract thereof, about 0.1%-0.3% opuntia ficus-indica or an extract thereof, and up to about 0.1% prunus persica gum; a composition of Table 2, comprising about 0.01%-0.1% alpha-glucan oligosaccharide, about 0.01%-0.1% kunzea pomifera or an extract thereof, about 0.01%-0.1% syzygium luehmannii or an extract thereof, and about 0.01%-0.1% tasmannia lanceolata or an extract thereof or a composition of Table 1, comprising about 0.1%-0.3% sarcosine, about 0.01%-0.1% of kunzea pomifera or an extract thereof, about 0.01%-0.1% of syzygium luehmannii or an extract thereof, about 0.01%-0.1% of tasmannia lanceolata or an extract thereof, about 0.01%-0.1% of pisum sativum or an extract thereof, and about 0.01%-0.1% of salvia hispanica or an extract thereof. Bacterial colonies were counted and normalized to control. Results for the composition of Table 3, the composition of Table 2, and the composition of Table 1 are shown in FIGS. 1A-1C, respectively.

The S. aureus, opportunistic pathogen, bacterial count after 15 minutes of incubation with the composition was reduced to >30% of the control bacterial count in all compositions. The growth of S. epidermidis, scalp commensal, was reduced compared to the control (100%) for the composition of Table 1 and composition of Table 2, showing that these two compositions can modulate the growth of resident microbes on the scalp without completely disrupting their presence, while also maintaining a higher ratio % of S. epidermidis to S. aureus.

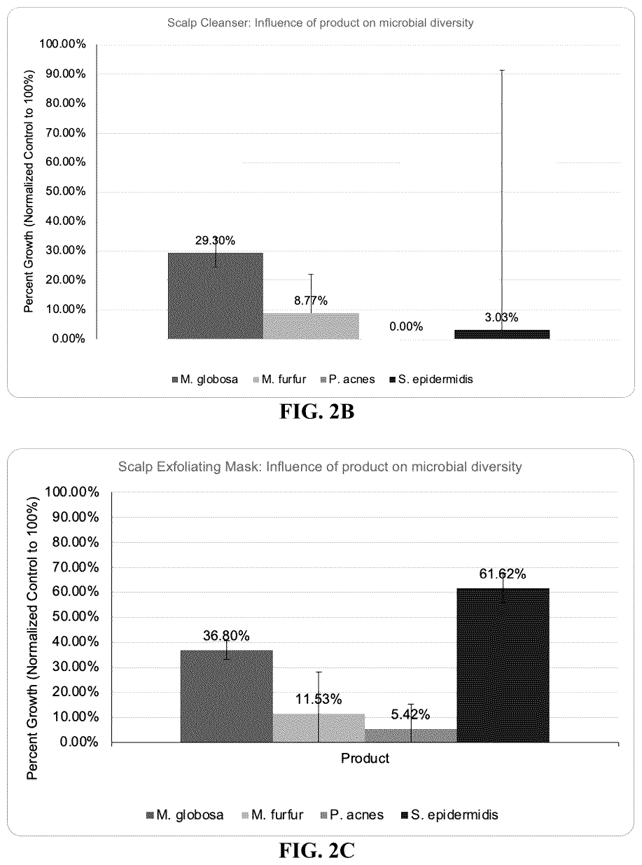

A co-culture of Malassezia globosa, Malassezia furfur, Propionibacterium acnes, and S. epidermis was incubated with a composition of Table 3, a composition of Table 2, or a composition of Table 1 for 15 minutes. Bacterial colonies were counted and normalized to control. The co-culture allows the microbes to interact with each other, mimicking scalp conditions. Results for the composition of Table 3, the composition of Table 2, and the composition of Table 1 are shown in FIGS. 2A-2C, respectively.

The composition of Table 1 and composition of Table 2 rebalanced the microbial diversity by suppressing, without eradicating, P. acnes, an opportunistic pathogen that is associated with acne, and the Malassezia yeasts that are associated with dandruff conditions. The compositions were also able to maintain the growth of S. epidermidis, a scalp commensal that produces beneficial metabolites. The composition of Table 3 selectively maintained the growth of scalp commensals (S. epidermidis), gently suppressed the growth of opportunistic pathogen, P. acnes, and did not impact the Malassezia species, effectively restoring the microbial growth.

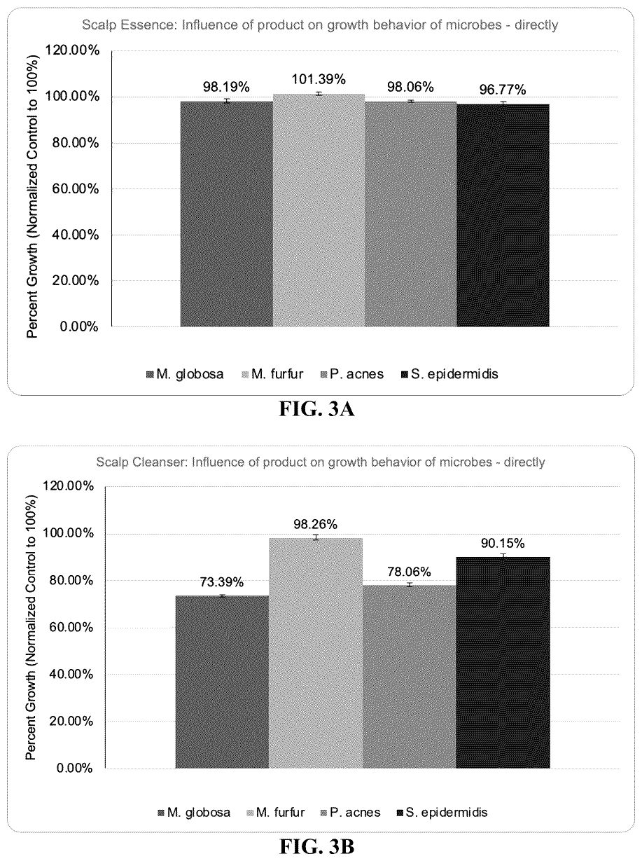

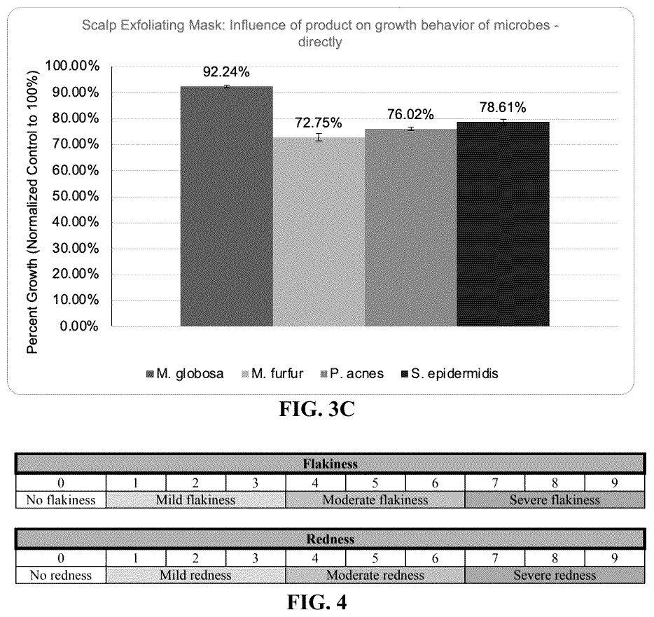

To determine whether the compositions have bactericidal effects, a composition of Table 3, a composition of Table 2, or a composition of Table 1 was applied directly to an over-abundant agar plate (>1000 colonies) of an individual microbe (Malassezia globosa, Malassezia furfur, Propionibacterium acnes, or S. epidermis), and the bacterial growth post-application was quantified. Results for the composition of Table 3, the composition of Table 2, and the composition of Table 1 are shown in FIGS. 3A-3C, respectively. The effect of each composition on the microbes is not antibacterial or inhibitory (since not all bacterial growth was stopped), but rather, the products sensitively modulated bacterial growth.

### Example 2: A Clinical Study to Evaluate the Efficacy and Consumer Perception of a Composition of Table 3, a Composition of Table 2, and a Composition of Table 1

This example describes a clinical study to assess the changes to the scalp after use of a test product (a composition of Table 2 (“scalp shampoo”), comprising about 0.01%-0.1% alpha-glucan oligosaccharide, about 0.01%- 0.1% kunzea pomifera or an extract thereof, about 0.01%-0.1% syzygium luehmannii or an extract thereof, and about 0.01%-0.1% tasmannia lanceolata or an extract thereof; a composition of Table 1 (“exfoliating mask”), comprising about 0.1%-0.3% sarcosine, about 0.01%-0.1% of kunzea pomifera or an extract thereof, about 0.01%-0.1% of syzygium luehmannii or an extract thereof, about 0.01%-0.1% of tasmannia lanceolata or an extract thereof, about 0.01%-0.1% of pisum sativum or an extract thereof, and about 0.01%-0.1% of salvia hispanica or an extract thereof; or a composition of Table 3 (“scalp essence”), comprising about 1%-3% alpha-glucan oligosaccharide, about 0.1%-0.3% bacillus ferment filtrate or an extract thereof, about 0.1%-0.3% opuntia ficus-indica or an extract thereof, and up to about 0.1% prunus persica gum) and to determine consumer perception of the test product through the use of questionnaires.

Study Design

Three groups of approximately 40 subjects were enrolled to assess the changes to the scalp after use of a test product, obtain images of the scalp, obtain microbiome swabs of the scalps of randomly selected subjects from each group, and obtain consumer perception of the test product through the use of questionnaires. Approximately 40 volunteers tested one test product (a scalp shampoo, an exfoliating mask, or a scalp essence). In total, 33 subjects completed the study for the scalp essence, 38 subjects completed the study for the exfoliating mask and 37 subjects completed the study for the scalp shampoo.

Product Use Instructions

The following instructions were provided to subjects for assessment of the scalp shampoo: “Apply a dollop to fingertips. Use enough to cover the full scalp. Massage into the scalp for 2 minutes, then rinse thoroughly with water. Repeat as needed. Please use according to your regular schedule. Minimum of 3× per week.”

The following instructions were provided to the subjects for assessment of the scalp essence: “Apply nozzle directly to the scalp. Lightly squeeze the bottle while sectioning the hair into 6 sections from the front to the back with the top of the bottle. Do not rinse. For best results use daily.”

The following instructions were provided to the subjects for assessment of the exfoliating mask: “With the tip of the tube, section the hair and apply mask directly to scalp in 6 sections. Use enough mask to lightly cover the desired area, then massage into the scalp with fingertips. The product should cover the entire scalp. Leave in for 3 to 5 minutes. Rinse with water. Use 2 times per week pre-shampoo”.

Inclusion and Exclusion Criteria:

Inclusion Criteria for Scalp Shampoo Group:
a) Subject is female between 18 and 60 years of age (attempt will be made to evenly distribute subjects in their 20s, 30s, 40s, 50+);
b) Subject has oily scalp (approximately 50% of the study population) or has dry scalp (approximately 50% of the study population) as confirmed by cosmetologist;
c) Subject has self-perceived sensitive skin/scalp (100% of the study population);
d) Subject has mild to moderate flaky scalp (can be from either dry scalp or oily scalp with sebum buildup scales) as confirmed by cosmetologist;
e) Subject has color treated hair (approximately 50% of the study population) or non-color treated hair (approximately 50% of the study population);
f) Subject agrees to use current normal conditioner throughout the entirety of the study;
g) Subject agrees not to introduce any new cosmetic or toiletry products during the study, like new/different conditioner;
h) Subject is willing to maintain their normal washing schedule;
i) Subject agrees not to dye hair throughout the duration of the study.

Inclusion Criteria for the Scalp Essence Group:
a) Subject is female between 18 and 60 years of age;
b) Subject must have moderately dry, flaky, and irritated (redness) scalp, as confirmed by the cosmetologist;
c) Subjects is willing to continue use of their regular shampoo and conditioner;
d) Subject agrees not to dye hair and keep the same hair style throughout the duration of the study;
e) Subject has self-perceived thinning hair;
f) Subject has a self-perceived sensitive scalp (100%);
g) Subject must have color treated hair (at least 50% of the population);
h) Subject agrees not to introduce any new cosmetic or toiletry products to the testing site during the study, like shampoo and/or conditioner.

Inclusion Criteria for Exfoliating Mask Group:
a) Subject is female between 18 and 60 years of age;
b) Subject must have moderate to high amount of sebum on scalp, as confirmed by a cosmetologist;
c) Subject has self-perceived moderate amount of yellowish-white sebum build-up when hair has not been washed for 24 hours;
d) Subject must have color treated hair or non color treated hair (minimum of 50% of
e) the population with color treated hair and a maximum of 50% of the population have non color treated hair);
f) Subject has a self-perceived sensitive scalp (100%);
g) Subject agrees to bring in their shampoo and conditioner at the Baseline study visit;
h) Subject agrees to wash their own hair at ECRL for the Baseline Visit;
i) Subject agrees not to introduce any new cosmetic or toiletry products to the testing site, during the study, like shampoo or conditioner.

Exclusion Criteria (for all Groups)
a) Subject is pregnant, nursing, planning a pregnancy, or not using adequate birth control;
b) Subject is currently using or has used in the past week any systemic or topical corticosteroids, nonsteroid anti-inflammatory drugs, antihistamines, sympathomimetics, and/or vasoconstrictors;
c) Subject has known allergies to skin treatment products or cosmetics, toiletries, and/or topical drugs;
d) Subject has a history of acute or chronic dermatologic, medical, and/or physical conditions which would preclude application of the test material and/or could influence the outcome of the study;
e) Subject exhibits irritation, tattoos, sunburn, rashes, scratches, burn marks, scarring, etc. on the test sites, which might interfere with evaluations of test results;
f) Subject is currently taking any medications which may interfere with the study;
g) Subject has a history of skin cancer, or is currently undergoing treatment for active cancer of any kind;
h) Subject has diabetes.

Study Evaluations

Corneometer

Three Corneometer measurements were obtained from the top center of the scalp when the hair is sectioned down the middle at each designated time point.

Sebumeter

One Sebumeter measurement was obtained from the top center of the scalp when the hair is sectioned down the middle at each designated time point using a Sebumeter SM 810 PC.

pH Measurements

One pH meter measurement was obtained from the top center of the scalp when the hair is sectioned down the middle at each designated time point using a Skin-pH-Meter PH 905.

FotoFinder Medicam

Images were captured in the Trichogram mode and data generated using the Trichoscale program. The following parameters were collected for analysis: “Overview” images of the scalp were captured with Medicam 1000 and up-close images of the scalp (when hair is sectioned down the middle) were captured using Medicam 1000+D-Scope IV 20×.

Expert Clinical Grading

Clinical grading from images of the scalp (when sectioned down the middle) was performed by a cosmetologist for the following flakiness and redness parameter using the scoring scale shown in FIG. 4.

Hair Count after Combing

Each subject's hair was divided into four quadrants; front left, front right, back left, and back right. If hair length was not sufficient enough to be parted in order to be divided, the hair was be combed to form the four quadrants without being divided. The subjects combed the hair onto a white paper, applying two strokes to each quadrant, for a total of eight strokes. Hairs that fell onto the white surface were counted.

Swab Kits

One swab sample from the center of the scalp (when parted down the middle) for 15 subjects (randomized) was obtained at each designated time point, stored at −80° C. and processed for sequencing analysis. The microbiome composition on the scalp was sequenced to examine the effect of the testing product on viability and colonization.

Consumer Perception Questionnaire

An assessment of test material attributes and the effects of a test material was determined by questioning the treated subject with regard to consumer perception and the efficacy of the test material following use. Consumer Perception Questionnaires could be answered with “Strongly Agree,” “Agree,” “Disagree,” “Strongly Disagree,” or “Not Applicable.” Questionnaires for the consumer perception of the scalp shampoo, the scalp essence and the exfoliating mask are shown in Table 4, Table 5, and Table 6, respectively:

TABLE 4
Scalp Shampoo Assessment Questionnaire

| | |
|---|---|
| 1) | My scalp feels more hydrated |
| 2) | My scalp feels more nourished. |
| 3) | My scalp is deep cleansed. |
| 4) | My scalp feels less congested. |
| 5) | My oil production is balanced. |
| 6) | My scalp feels revitalized. |
| 7) | My scalp is soothed. |
| 8) | Reduces the appearance of dryness and flakiness on my scalp. |
| 9) | Reduces the appearance of redness on my scalp. |
| 10) | My scalp feels less itchy. |
| 11) | My hair feels more hydrated. |
| 12) | My hair feels more nourished. |
| 13) | My hair looks shinier. |
| 14) | My hair feels softer. |
| 15) | My hair texture improved. |
| 16) | My hair feels stronger. |
| 17) | My hair looks more voluminous. |
| 18) | My hair looks/feels healthier. |
| 19) | My hair looks younger. |
| 20) | This product has good lather. |
| 21) | I would replace my current shampoo for this product. |
| 22) | I would recommend this shampoo to a friend. |

TABLE 5
Scalp Essence Assessment Questionnaire

| | |
|---|---|
| 1) | My scalp feels more hydrated. |
| 2) | My scalp feels more nourished. |
| 3) | My oil production is balanced. |
| 4) | My scalp is soothed. |
| 5) | Reduces the appearance of dryness and flakiness on my scalp. |
| 6) | Reduces the appearance of redness on my scalp. |
| 7) | My scalp feels less itchy. |
| 8) | My scalp feels less irritated. |
| 9) | My hair feels more hydrated. |
| 10) | My hair feels more nourished. |
| 11) | My hair looks shinier. |
| 12) | My hair looks more radiant. |
| 13) | My hair feels softer. |
| 14) | The texture of my hair has improved. |
| 15) | My hair feels stronger. |
| 16) | My hair looks more voluminous. |
| 17) | My hair looks/feels healthier. |
| 18) | My hair looks younger. |
| 19) | This product was gentle on my scalp. |
| 20) | I would add this product into my regular routine. |
| 21) | I would recommend this product to a friend. |
| 22) | I was surprised by how much my hair improved by adding this serum. |

TABLE 6
Exfoliating Mask Assessment Questionnaire

| | |
|---|---|
| 1) | My scalp is cleansed deeply. |
| 2) | My scalp feels less congested. |
| 3) | My scalp feels detoxed. |
| 4) | My scalp is renewed. |
| 5) | My oil production is balanced. |
| 6) | Excess build up on my scalp is dissolved. |
| 7) | My scalp is less dry and flakey. |
| 8) | My scalp is less itchy. |
| 9) | My hair is more hydrated. |
| 10) | My hair feels more nourished. |
| 11) | My hair looks shinier. |
| 12) | My hair feels softer. |
| 13) | My hair texture bas improved. |
| 14) | My hair feels stronger |
| 15) | My hair looks more voluminous. |
| 16) | My hair looks healthier. |
| 17) | My hair looks younger. |
| 18) | This product was gentle on my scalp. |
| 19) | I would add this product into my regular routine. |
| 20) | I would recommend this shampoo to a friend. |

Test Method

The following protocol was used for the three groups:

Scalp Shampoo Group:

Screening: Subjects were instructed to report to the testing facility having not washed their hair for 24 to 36 hours previously. Informed consent was obtained. Inclusion and exclusion criteria were verified. Subjects were asked to keep using their own shampoo and conditioner and discontinue use of any other hair care products. Subjects were instructed to bring in their shampoo and conditioner at Baseline.

Baseline: Subjects returned to the testing facility having not washed their hair for 24 to 36 hours previously. Subjects were allowed to acclimate to ambient laboratory conditions for approximately 15 minutes (±5 minutes). The following study evaluations were performed: FotoFinder Medicam 1000 images, Sebumeter measurements, pH measurements, Corneometer measurements. An ECRL Cosmetologist washed the subject's hair with the scalp shampoo test product and applied the subject's conditioner in the lab. The Cosmetologist then blow-dried the subject's hair.

Immediately Post-Application/Blow Drying (±20 minutes): Following test material application and post 20 minutes blow drying, the following study evaluations were performed: FotoFinder Medicam 1000 images, Sebumeter measurements, pH measurements, Corneometer measurements, and consumer perception questionnaire. Test materials, use instructions, and daily diaries were distributed.

Day 7: Subjects returned to the testing facility having not washed their hair for 24 to 36 hours previously. Subjects were allowed to acclimate to ambient laboratory conditions for approximately 15 minutes (±5 minutes). The following study evaluations were performed: FotoFinder Medicam 1000 images, Sebumeter measurements, pH measurements, Corneometer measurements, and consumer perception questionnaire.

Day 14 (Final Visit): Subjects returned to the testing facility having not washed their hair for 24 to 36 hours previously. Used and unused test materials were collected. Daily diaries were reviewed for study compliance and collected. Subjects were allowed to acclimate to ambient laboratory conditions for approximately 15 minutes (±5 minutes). The following study evaluations were performed: FotoFinder Medicam 1000 images, Sebumeter measurements, pH measurements, Corneometer measurements, and consumer perception questionnaire.

Scalp Essence Group:

Screening: Subjects were instructed to report to the testing facility. Informed consent was obtained. Inclusion and exclusion criteria were verified. Subjects were asked to keep using their own shampoo and conditioner and discontinue use of any other hair care products.

Baseline: Subjects returned to the testing facility. Subjects were allowed to acclimate to ambient laboratory conditions for approximately 15 minutes (±5 minutes). The following study evaluations were performed: FotoFinder Medicam 1000 images, Sebumeter measurements, pH measurements, Corneometer measurements, Swab of scalp of 15 randomized subjects. Assessment of hair retention was performed by an expert clinical grader. Test material, daily dairy and use instruction were distributed.

Day 7: Subjects returned to the testing facility. Subjects were allowed to acclimate to ambient laboratory conditions for approximately 15 minutes (±5 minutes). The following study evaluations were performed: FotoFinder Medicam 1000 images, Sebumeter measurements, pH measurements, Corneometer measurements, and consumer perception questionnaire. Assessment of hair retention was performed by an expert clinical grader.

Day 14: Subjects returned to the testing facility. Subjects were allowed to acclimate to ambient laboratory conditions for approximately 15 minutes (±5 minutes). The following study evaluations were performed: FotoFinder Medicam 1000 images, Sebumeter measurements, pH measurements, Corneometer measurements, and consumer perception questionnaire. Assessment of hair retention was performed by an expert clinical grader.

Day 28 (Final Visit): Subjects returned to the testing facility. Used and unused test materials were collected. Daily diaries were reviewed for study compliance and collected. Subjects were allowed to acclimate to ambient laboratory conditions for approximately 15 minutes (±5 minutes). The following study evaluations were performed: FotoFinder Medicam 1000 images, Sebumeter measurements, pH measurements, Corneometer measurements, swab of scalp of 15 randomized subjects, and consumer perception questionnaire. Assessment of hair retention was performed by an expert clinical grader.

Exfoliating Mask Group:

Screening: Subjects were instructed to report to the testing facility having not washed their hair for 24 to 36 hours previously. Informed consent was obtained. Inclusion and exclusion criteria were verified. Subjects were asked to keep using their own shampoo and conditioner and discontinue use of any other hair care products. Subjects were instructed to bring in their shampoo and conditioner at Baseline.

Baseline: Subjects returned to the testing facility having not washed their hair for 24 to 36 hours previously. Subjects were allowed to acclimate to ambient laboratory conditions for approximately 15 minutes (±5 minutes). The following study evaluations were performed: FotoFinder Medicam 1000 images, Sebumeter measurements, pH measurements, Swab of scalp of 15 randomized subjects. Expert clinical grading of sebum build-up will be done from photos. Under the supervision of an ECRL technician the subjects shampooed and conditioned (with their own hair products) their hair in the lab. They then applied the exfoliating mask, and blow-dried their own hair.

Immediately Post-Application/Blow Drying (±20 minutes): Following test material application and post 20 minutes blow drying, the following study evaluations were performed: FotoFinder Medicam 1000 images, Sebumeter measurements, pH measurements, and consumer perception questionnaire. Expert clinical grading of sebum build-up was done from photos. Test materials, use instructions, and daily diaries were distributed.

Day 7: Subjects returned to the testing facility having not washed their hair for 24 to 36 hours previously. Subjects were allowed to acclimate to ambient laboratory conditions for approximately 15 minutes (±5 minutes). The following study evaluations were performed: FotoFinder Medicam 1000 images, Sebumeter measurements, pH measurements, and consumer perception questionnaire. Expert clinical grading of sebum build-up was done from photos.

Day 14: Subjects returned to the testing facility having not washed their hair for 24 to 36 hours previously. Subjects were allowed to acclimate to ambient laboratory conditions for approximately 15 minutes (±5 minutes). The following study evaluations were performed: FotoFinder Medicam 1000 images, Sebumeter measurements, pH measurements, and consumer perception questionnaire. Expert clinical grading of sebum build-up was done from photos.

Day 28 (Final Visit): Subjects returned to the testing facility having not washed their hair for 24 to 36 hours previously. Used and unused test materials were collected. Daily diaries were reviewed for study compliance and collected. Subjects were allowed to acclimate to ambient laboratory conditions for approximately 15 minutes (±5 minutes). The following study evaluations were performed: FotoFinder Medicam 1000 images, Sebumeter measurements, pH measurements, Swab of scalp of 15 randomized subjects, and consumer perception questionnaire. Expert clinical grading of sebum build-up will be done from photos.

Data Tabulation

Analysis of variance followed by Dunnett's test was applied to determine the differences between baseline and each post-treatment interval for the following parameters: FotoFinder Medicam 1000 images, Corneometer measurements, Sebumeter measurements, pH Meter measurements, Clinical grading from images of the scalp and Assessment of Hair Retention. Change from baseline and % of subjects improved were calculated at each post- treatment interval for the above-mentioned parameters.

Statistical significance exists for two-sided p-values≤0.05. Questionnaire responses, for which response category comparisons are informative, were analyzed by Z-tests. Z-tests are used to determine statistically significant differences in the proportions of subjects responding positively or negatively to each question offering a range of responses. If applicable, the proportions of subjects choosing the central (neutral) responses were split equally and added to the response proportion of the top and bottom choices. The split proportions were compared by calculation of a Z-Score to determine statistically significant differences. Statistical significance exists for Z-scores greater than or equal to the absolute value of 1.96 at the 95% confidence level. Swabs were stored at −80 C and shipped to a third-party lab for microbiome analysis.

Adverse Events

An adverse event is any untoward medical occurrence, whether or not it is considered study-related, including death, experienced by a subject. An event may consist of a disease, an exacerbation of a pre-existing illness or condition, an occurrence of an intermittent illness or condition, a set of related symptoms or signs, or a single symptom or sign. A serious adverse event is any event in which the subject is, at immediate risk of death or persistent or significant incapacity or substantial disruption of the ability to conduct normal life functions.

All adverse events were promptly recorded. All adverse events, serious or not serious, related or not related to the test material, were summarized and reported in the final report. All adverse events were followed up until resolved, stabilized, the subject was lost to follow-up, or the event was otherwise explained. Any report of dermal irritation or sensory experiences was listed as experimental data in the clinical study report, and was not considered an adverse event unless confirmed in the laboratory by medical staff.

Results

Scalp Shampoo Group

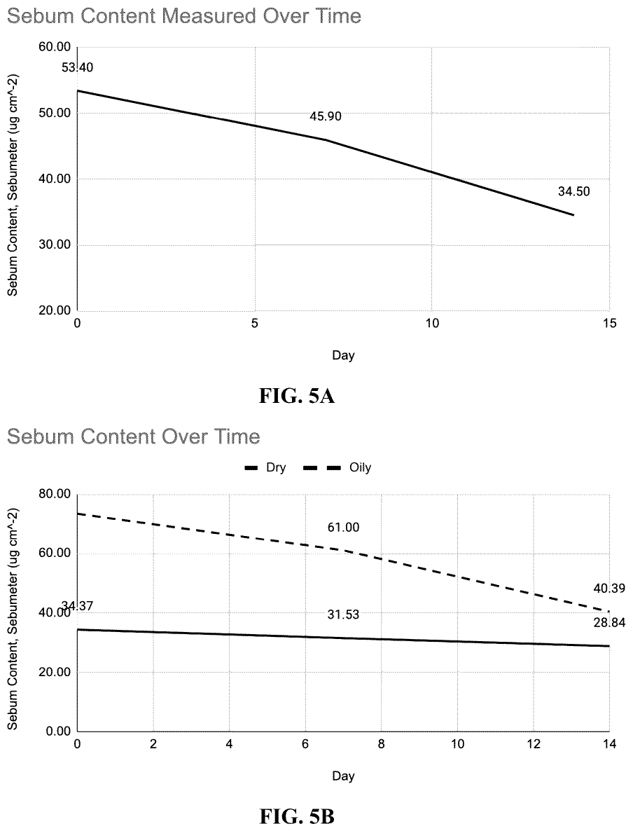

After 14 days of scalp shampoo use, subjects' sebum content decreased significantly (18.9%, p<0.001) (FIG. 5A). Dry group (baseline sebum<50) sebum levels decreased 16% over the course of 14 days using scalp shampoo. Oily group (baseline sebum>50) decreased 44% over the course of 14 days using scalp shampoo (FIG. 5B).

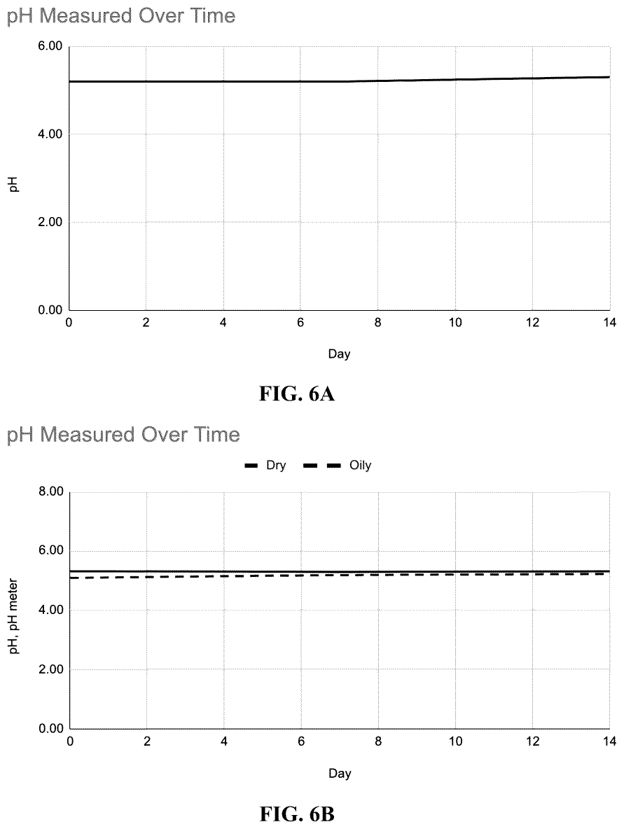

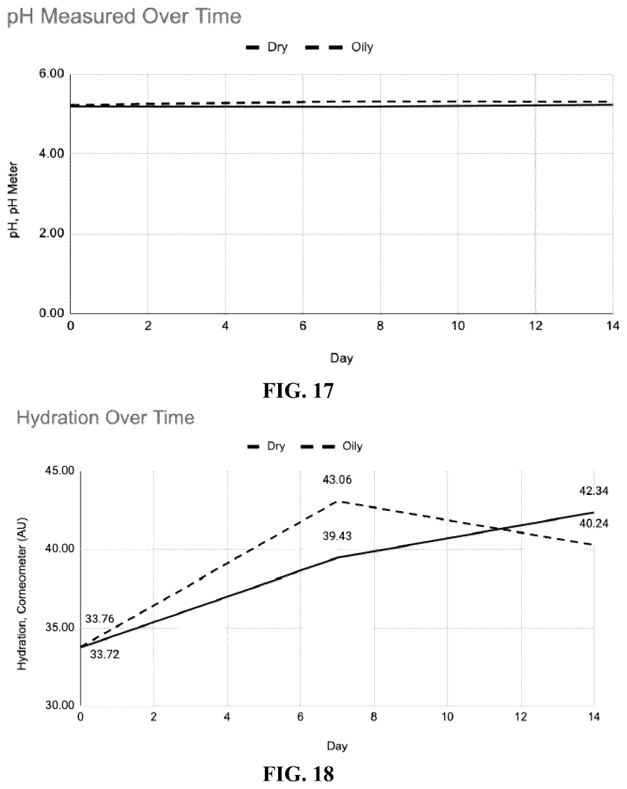

Subjects' scalps did not significantly change pH (p>0.05), showing that the scalp shampoo maintains pH over time (FIG. 6A). This was observed in both the oily group and the dry group (FIG. 6B). FIG. 17 displays the change in pH over the course of 14 days of scalp shampoo use for the dry and oily groups; dry and oily group determined by Expert Evaluation. There was no change in scalp pH with use of the scalp shampoo (% change<2% overall).

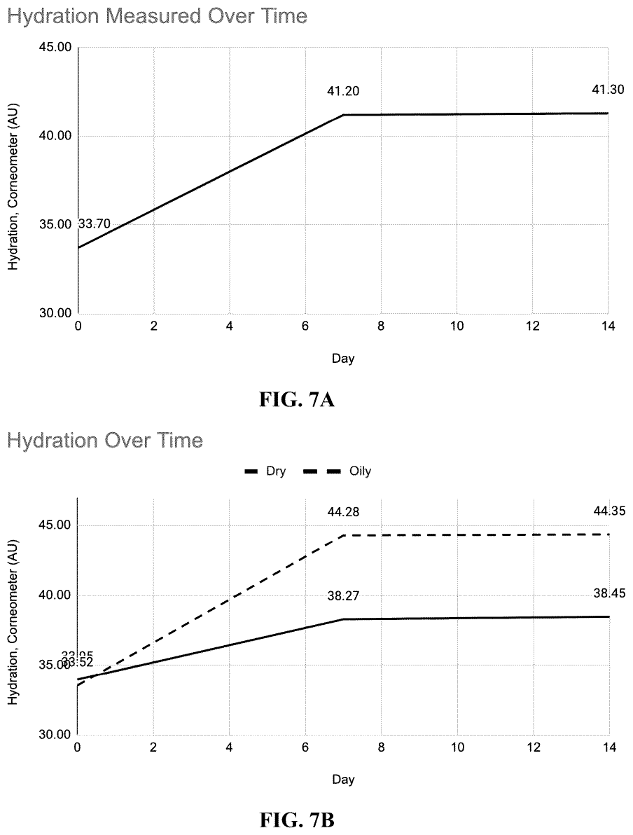

Scalp hydration, measured by corneometer, significantly improved from baseline by 7.5% after 7 days (p<0.01) and by 7.6% after 14 days (p<0.01) (FIG. 7A). The dry scalp group hydration improved 13% over the course of 14 days of scalp shampoo use, while the oily scalp group improved 32% over the course of 14 days of scalp shampoo use (FIG. 7B). FIG. 18 shows the scalp hydration, as measured by Corneometer, over time (14 days) with use of scalp shampoo for the dry and oily groups; dry and oily scalp groups determined by Expert Evaluator. The dry scalp group hydration improved 25% over the course of 14 days of scalp shampoo use. The oily scalp group improved 19% over the course of 14 days of scalp shampoo use.

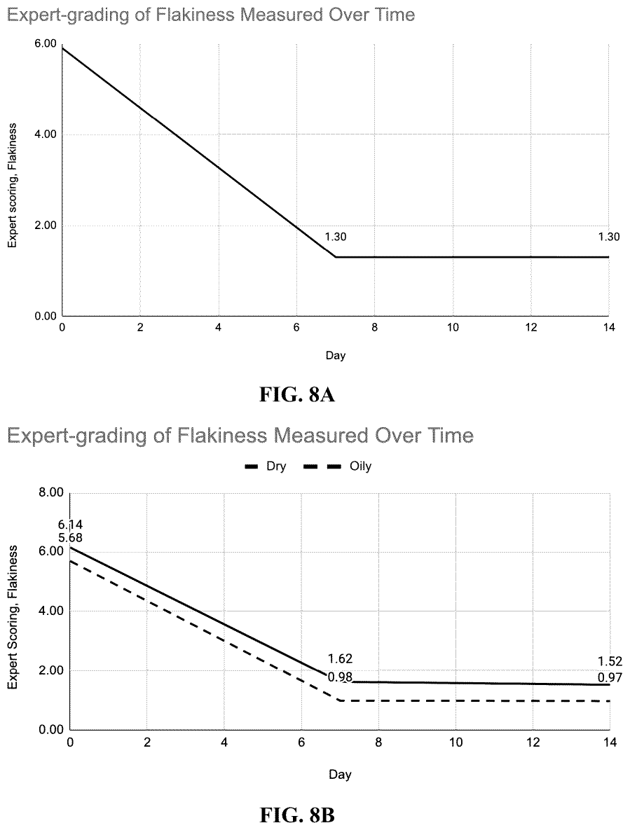

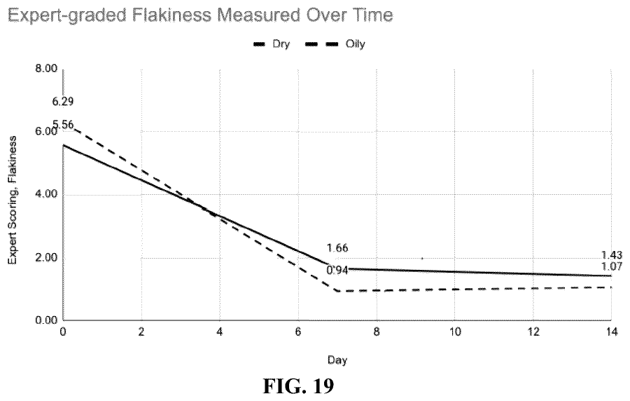

Subjects' scalp flakiness, as measured by Expert Scoring via Dermatologist, with use of scalp shampoo significantly improved from baseline by 4.6% after 7 days (p<0.0001) and by 4.7% (p<0.0001) after 14 days (FIG. 8A). The dry group improved 75% and the oily group 82% in expert-graded scalp flakiness scoring after 14 days of using scalp shampoo (FIG. 8B). FIG. 16 depicts the sebum content over time for the dry group and the oily group; dry and oily status determined by an Expert evaluator. Dry group sebum levels decreased 32% over the course of 14 days using scalp shampoo. Oily group (baseline sebum>50) decreased 37% over the course of 14 days using scalp shampoo. FIG. 19 shows scalp flakiness as measured by an Expert Scorer (Dermatologist) over the course of 14 days using scalp shampoo for the dry and oily groups; dry and oily groups determined by Expert Evaluator. The dry group improved 74% and the oily group 83% in expert-graded scalp flakiness scoring after 14 days of using scalp shampoo.

Exfoliating Mask

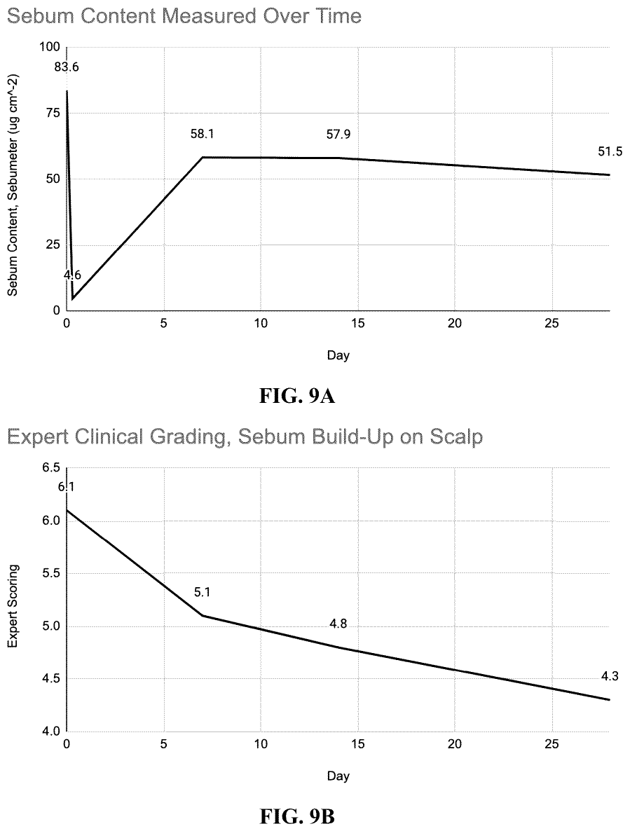

Subjects' scalp sebum measurements as determined by sebumeter changed decreased 94% from baseline immediately after use of exfoliating mask (p<0.0001), 30% after 7 days (p=0.02), 30% after 14 days (p=0.02), and 38% after 28 days (p=0.002) (FIG. 9). 100% of subjects improved immediately, 55.3% of subjects improved after Day 7, 55.3% of subjects improved after Day 14, 65.8% of subjects improved after Day 28. Sebum build-up scores as determined by expert clinical grading decreased over 28 days of use: 16.4% after 7 days (p<0.0001), 21.3% after 14 days (p<0.0001), and 29.5% after 28 days (p<0.0001). 97.4% of subjects improved immediately, 76.3% of subjects improved after Day 7, 78.9% of subjects improved after Day 14, 92.1% of subjects improved after Day 28. Table 7 shows the Questionnaire Results, Consumer Self-perceived Results for the exfoliating mask. Only reported statistically significant questionnaire responses, as evaluated by Z test scores.

TABLE 7
Questionnaire Results, Consumer Self-perceived Results for the Exfoliating Mask.

| Questionnaire Z test Day 14 | Questionnaire Z test Day 28 |
|---|---|
| Z test = Significant | Z test = Significant |
| 1) My scalp is cleansed deeply. 97.4% | 1) My scalp is cleansed deeply. 94.7% |
| 2) My scalp feels less congested. 89.5% | 2) My scalp feels less congested. 84.2% |
| 3) My scalp feels detoxed. 86.8% | 3) My scalp feels detoxed. 89.5% |
| 4) My scalp is renewed. 86.8% | 4) My scalp is renewed. 86.8% |
| 5) My oil production is balanced. 89.5% | 5) My oil production is balanced. 81.6% |
| 6) Excess build up on my scalp is dissolved. | 6) Excess build up on my scalp is dissolved. |
| 86.5% | 73.7% |
| 7) My scalp is less dry and flakey. 83.8% | 7) My scalp is less dry and flakey. 83.3% |
| 8) My scalp is less itchy. 88.2% | 8) My scalp is less itchy. 88.9% |
| 9) My hair is more hydrated. 86.8% | 9) My hair is more hydrated. 92.1% |
| 10) My hair feels more nourished. 92.1% | 10) My hair feels more nourished. 89.5% |
| 11) My hair looks shinier. 84.2% | 11) My hair looks shinier. 78.9% |
| 12) My hair feels softer. 84.2% | 12) My hair feels softer. 86.8% |
| 13) My hair texture has improved. 84.2% | 13) My hair texture has improved. 73.7% |
| 14) My hair feels stronger. 78.9% | 14) My hair feels stronger. 73.7% |
| 15) My hair looks more voluminous. 81.6% | 15) My hair looks more voluminous. 73.7% |
| 16) My hair looks healthier. 89.5% | 16) My hair looks healthier. 86.8% |
| 17) My hair looks younger. 76.3% | 17) My hair looks younger. 76.3% |
| 18) This product was gentle on my scalp. 94.7% | 18) This product was gentle on my scalp. 92.1% |
| 19) I would add this product into my regular | 19) I would add this product into my regular |
| routine. 94.7% | routine. 86.5% |
| 20) I would recommend this shampoo to a | 20) I would recommend this shampoo to a |
| friend. 94.7% | friend. 91.7% |

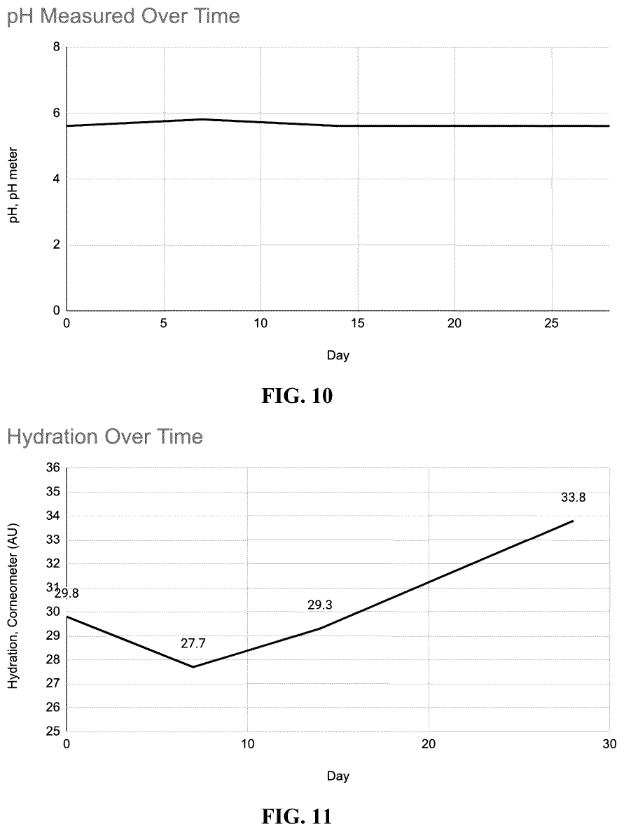

The scalp pH did not significantly change over a month of exfoliating mask use on day 7 (p>0.05), day 14 (p>0.05), or day 28 (p>0.05) (FIG. 10).

Scalp Essence

There was no significant improvement of hydration after 28 days of using scalp essence, but a trending increase in hydration was observed. After 28 days, 60.6% of subjects improved hydration (FIG. 11).

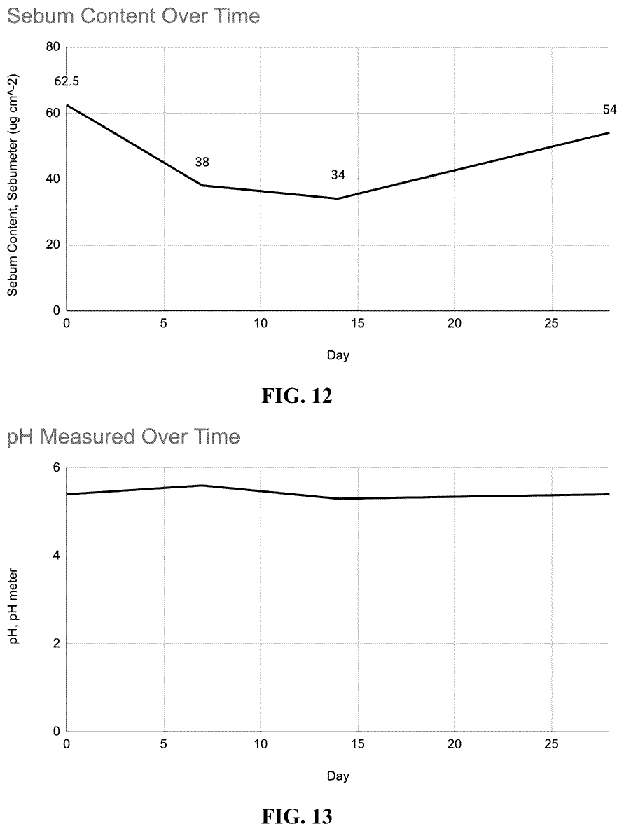

After 14 days of use, sebum content, as measured by sebumeter, decreased by 45.6% from baseline (p=0.02), however, at day 28, there was no significant improvement compared to baseline, showing that sebum levels regulated back to baseline conditions (FIG. 12). After 7 days, 72.7% of subjects showed reduced sebum content; after 14 days, 14 72.7% of subjects showed reduced sebum content; and after 28 days, 66.7% of subjects showed reduced sebum content.

No significant change in pH (p>0.05) after 28 days of using scalp essence was observed (FIG. 13), indicating that the scalp pH is maintained.

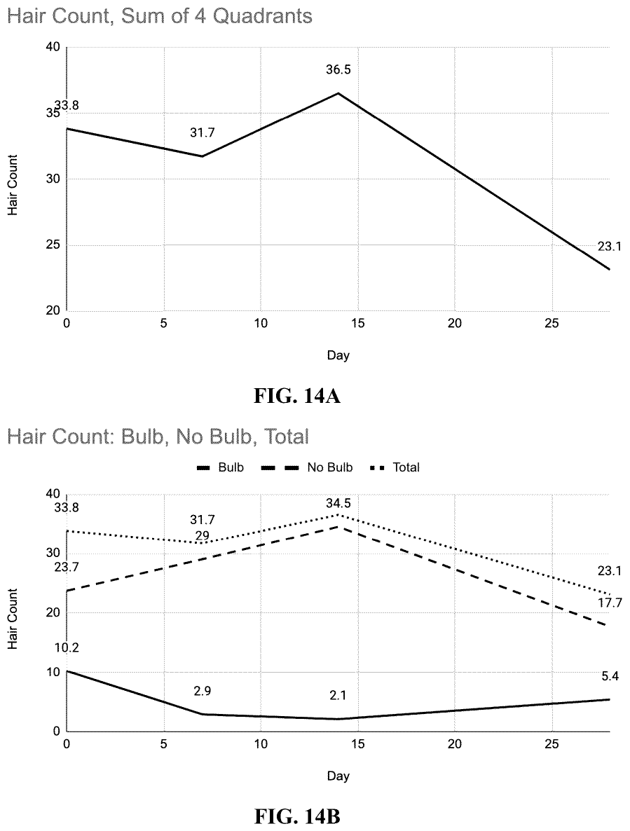

Shedding of hair, as measured by the hair count sum of 4 quadrants collected from a brush, improved significantly after 28 days of using the scalp essence (31% decrease compared to baseline; p=0.04) (FIG. 14A). After 28 days, hair count (sum of 4 quadrants) improved for 60.6% of subjects. The count of hairs with bulbs found improved after 7 days (71%, p<0.001), 14 days (79%, p<0.001), and 28 days (47%, p<0.05) (FIG. 14B). The count of hairs without bulbs (broken hairs) improved after 14 days of using the essence (45%, p<0.05) and overall the total hairs counted improved after 28 days of using the scalp essence (31%, p<0.05) (FIG. 14B). 72.7% of subjects improved after day 7, 69.7% of subjects improved after day 14, 63.6% of subjects improved after day 28.

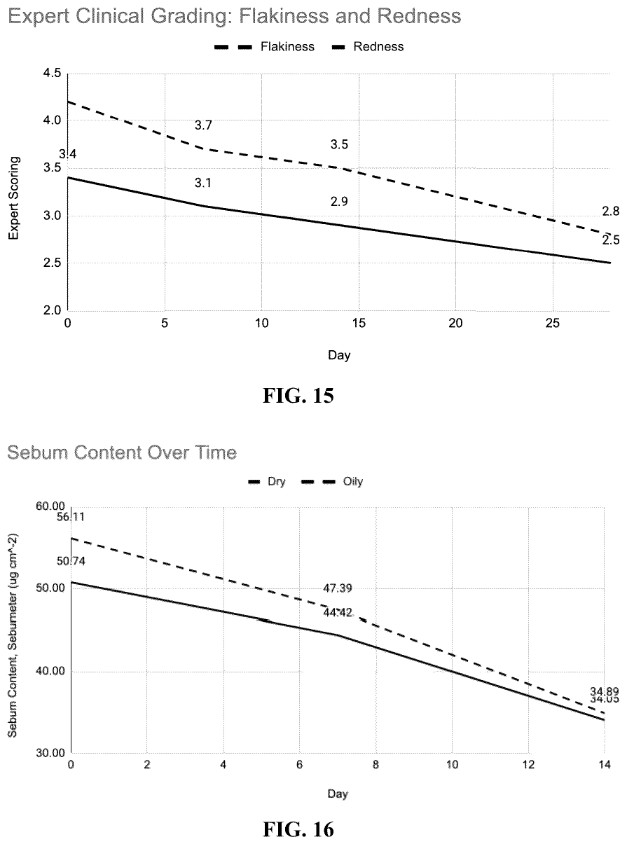

Flakiness, as determined by expert clinical grading, improved after 28 days of using the scalp essence (33%, p=0.0016) (FIG. 15). 66.7% of subjects improved flakiness (expert clinical grading) after 18 days. Similarly, redness, as determined by expert clinical grading, improved after 28 days of using scalp essence (26.5% p=0.0004) (FIG. 15). 60.6% of subjects improved redness (expert clinical grading) after 28 days.

Table 8 shows Questionnaire Results, Consumer Self-perceived Results for scalp essence. Only reporting statistically significant questionnaire responses, as evaluated by Z test scores.

TABLE 8
Questionnaire Results, Consumer Self-
perceived Results for Scalp Essence.
Questionnaire Z test Day 28

1. My scalp feels more hydrated. 87.9%
2. My scalp feels more nourished. 84.8%
3. My oil production is balanced. 84.8%
4. My scalp is soothed. 97.0%
5. Reduces the appearance of dryness and flakiness on my scalp. 75.8%
6. Reduces the appearance of redness on my scalp. 87.9%
7. My scalp feels less itchy. 90.9%
8. My scalp feels less irritated. 87.9%
9. My hair feels more hydrated. 84.8%
10. My hair feels more nourished. 81.8%
11. My hair looks shinier. 75.8%
12. My hair looks more radiant. 75.8%
13. My hair feels softer. 78.8%
14. The texture of my hair has improved. 75.8%
15. My hair feels stronger. 69.7%
17. My hair looks/feels healthier. 78.8%
19. This product was gentle on my scalp. 100%
20. I would add this product into my regular routine. 81.8%
21. I would recommend this product to a friend. 84.8%
22. I was surprised by how much my hair improved by adding this
serum. 78.8%

1. A composition comprising about 0.1%-0.3% w/w sarcosine, about 0.01%-0.1% w/w of kunzea pomifera or an extract thereof, about 0.01%-0.1% w/w of syzygium luehmannii or an extract thereof, about 0.01%-0.1% w/w of tasmannia lanceolata or an extract thereof, about 0.01%-0.1% w/w of pisum sativum or an extract thereof, about 0.01%-0.1% w/w of salvia hispanica or an extract thereof, about 0.1%-0.3% w/w of vaccinium myrtillus or an extract thereof, about 0.1%-0.3% w/w of saccharum officinarum (sugarcane) or an extract thereof, about 0.01%-0.1% w/w of citrus aurantium dulcis (orange) or an extract thereof, about 0.01%-0.1% w/w of citrus limon (lemon) or an extract thereof, about 0.01%-0.1% w/w of acer saccharum (sugar maple) or an extract thereof and about 1%-3% w/w of jojoba esters, Simmondsia chinensis or an extract thereof.

2.-4. (canceled)

5. The composition of claim 1, further comprising a cosmetically acceptable carrier or excipient.

6. (canceled)

7. A method of improving hair health or scalp health or treating or preventing hair loss in a subject in need of hair health or scalp health improvement or hair loss treatment or prevention, comprising applying to the scalp of the subject an effective amount of the composition of claim 1.

8.-11. (canceled)

12. A composition comprising about 0.01%-0.1% w/w alpha-glucan oligosaccharide, about 0.01%-0.1% w/w kunzea pomifera or an extract thereof, about 0.01%-0.1% w/w syzygium luehmannii or an extract thereof, about 0.01%-0.1% w/w tasmannia lanceolata or an extract thereof, about 0.01%-0.1% w/w of hydrolyzed quinoa and about 0.01%-0.1% w/w of aspergillus ferment.

13.-15. (canceled)

16. The composition of claim 12, further comprising a cosmetically acceptable carrier or excipient.

17. (canceled)

18. A method of improving hair health or scalp health or treating or preventing hair loss in a subject in need of hair health or scalp health improvement or hair loss treatment or prevention, comprising applying to the scalp of the subject an effective amount of the composition of claim 12.

19.-22. (canceled)

23. A composition comprising about 1%-3% w/w alpha-glucan oligosaccharide, about 0.1%-0.3% w/w bacillus ferment filtrate or an extract thereof, about 0.1%-0.3% w/w opuntia ficus-indica or an extract thereof, about 0.01%-0.1% w/w prunus persica gum, and about 1%-3% w/w smithsonite or an extract thereof.

24.-26. (canceled)

27. The composition of claim 23, further comprising a cosmetically acceptable carrier or excipient.

28. (canceled)

29. A method of improving hair health or scalp health or treating or preventing hair loss in a subject in need of hair health or scalp health improvement or hair loss treatment or prevention, comprising applying to the scalp of the subject an effective amount of the composition of claim 23.

30.-61. (canceled)
